(19) United States
(12) Patent Application Publication
(10) Pub. No.: US 2024/0209406 A1
(43) Pub. Date: Jun. 27, 2024

(54) ENZYMATIC SYNTHESIS OF NTP AND NQP

(71) Applicant: Codexis, Inc., Redwood City, CA (US)

(72) Inventors: David Entwistle, Dublin (IE); Stephanie Marie Forget, Redwood City, CA (US); Anders Matthew Knight, Mountain View, CA (US); Mikayla Jianghongxia Krawczyk, Palo Alto, CA (US); Marissa Greene MacAvoy, Emerald Hills, CA (US); Simon Ng, San Francisco, CA (US); Nicholas Porter, Redwood City, CA (US); Philip Provencher, San Francisco, CA (US); Amani Shoubber, Santa Clara, CA (US); Leann Quertinmont Teadt, Redwood City, CA (US); Jonathan Vroom, South San Francisco, CA (US); David Watts, San Mateo, CA (US); Leland Ken Wong, San Bruno, CA (US)

(21) Appl. No.: 18/486,140

(22) Filed: Oct. 12, 2023

Related U.S. Application Data
(60) Provisional application No. 63/387,908, filed on Dec. 16, 2022.

(51) Int. Cl. C12P 19/34; C12N 9/12

(52) U.S. Cl. CPC C12P 19/34; C12N 9/1205; C12N 9/1217; C12N 9/1223; C12N 9/1229; C12Y 207/0102; C12Y 207/02001; C12Y 207/03002; C12Y 207/04001; C12Y 207/04003; C12Y 207/04006

(57) ABSTRACT

The present disclosure provides enzymatic methods for the production of 3′-phosphate-nucleoside-5′-triphosphate (NQP). The present disclosure further provides enzymatic methods for the production of nucleotide-5′-triphosphates.

### CROSS-REFERENCE TO RELATED APPLICATIONS

This application claims the benefit of U.S. Provisional Application No. 63/387,908, filed Dec. 16, 2022, the entire contents of which is incorporated by reference herein.

### REFERENCE TO SEQUENCE LISTING, TABLE OR COMPUTER PROGRAM

The Sequence Listing concurrently submitted herewith as file name CX10-260WO1_ST26.xml, created on Oct. 12, 2023, with a file size of 9,199,375 bytes, is part of the specification and is hereby incorporated by reference herein.

### TECHNICAL FIELD

The present disclosure relates to an enzymatic method for producing 3′-phosphate-nucleoside-5′-triphosphate (NQP) nucleotides. The present disclosure further provides an enzymatic method for producing nucleotide-5′-triphosphates.

### BACKGROUND

Synthetic biology offers a diverse range of high value, high growth markets. From food and agriculture to therapeutics, diagnostics, and vaccines; tools such as gene editing, DNA sequencing and gene synthesis are being used to build value-added products with advanced functionality (e.g., cell bioreactors, etc.) and desired end products (e.g., drugs, chemicals, etc.). A barrier to widespread implementation of these technologies is the ability to efficiently synthesize RNA, DNA, and other polynucleotides.

In therapeutics, silencing RNA (siRNA) is a promising class of drugs that have the potential to treat numerous difficult to treat conditions in a highly targeted manner by binding to known mRNA targets (Hu et al., Sig Transduct Target Ther., 2020, 5:101; Zhang et al., Bioch. Pharmac., 2021, 189:114432). As these therapies become more common and are targeted at larger patient populations, the ability to produce large amounts of the oligonucleotide active pharmaceutical ingredient (API) becomes critical.

Another technology incorporating synthetic oligonucleotides is CRISPR (clustered, regularly interspaced, short palindromic repeats) technology, with increasing use of modified CRISPR RNAs, such as the guide RNA (gRNA) to improve activity and specificity for in vivo applications. Use of modified guide RNAs can reduce the levels of off-target activity (see, e.g., Hendel et al., Nat Biotechnol., 2015 September; 33(9): 985-989).

Phosphoramidite chemistry has been developed extensively over the years to synthesize small amounts of DNA and RNA, but suffers from several cost, processing and sustainability issues that are potentially limiting as API demand grows to triple-quadruple digit kilograms per year (see, e.g., Andrews et al., J. Org. Chem., 2021, 86:49-61). Additionally, RNA synthesis using phosphoramidite synthesis chemistry is limited to producing short oligonucleotides of approximately 200 base pairs (Beaucage and Caruthers, Tetrahedron Lett., 1981, 22 (20):1859).

New oligonucleotide synthesis techniques are being developed to replace to phosphoramidite chemistry to meet the growing demand for large quantities of DNA and RNA necessary for modern medical and industrial applications. One promising is template independent oligonucleotide synthesis using various polymerases, including terminal nucleotide transferases (TdTs) and polyX polymerases. These methods often rely on modified nucleotide triphosphates (NTPs) that incorporate blocking groups or other structural or chemical elements that allow the controlled addition of a defined sequence of NTPs. These modified NTPs include NTPs with blocking groups on the 3′ or 2′ positions of the sugar, as well as NTPs with modified bases or thiol derivates for the formation of more stable oligonucleotide phosphorothioate backbone bonds.

A limiting factor for the enzymatic oligonucleotide synthesis is the availability and cost of natural and modified NTPs as substrates for the TdTs and polyX polymerases. For example, NTPs with a phosphate at the 3′ position of the sugar (nucleoside tetraphosphates, pppNps or NQPs), with or without additional modifications to the nucleobase, sugar, and/or phosphate chain, are useful as reversible blocked nucleotide substrates for template independent enzymatic synthesis applications. However, these NQPs are not widely commercially available and associated with high costs at an industrial scale. Desirable are efficient methods to synthesize natural and modified NTPs and NQPs on the scale required for modern synthetic biology applications.

### SUMMARY

The present disclosure provides enzymatic methods and processes for producing 3′-phosphate-nucleoside-5-triphosphates. Further provided herein are enzymatic methods for producing nucleoside-5′-triphosphates.

In some embodiments, a method of producing a nucleotide triphosphate with a phosphate group at the 3′ position of the sugar moiety (NQP), the method comprising at least: contacting a nucleoside diphosphate (NDP) with a nucleoside diphosphate kinase, a 3′-O-kinase, and a phosphate donor under reaction conditions such that a NTP with a phosphate group on the 3′ position of the sugar moiety (NQP) is produced.

In some embodiments, the NDP substrate is an unmodified or modified NDP. In some embodiments, the NDP substrate is a nucleoside 5′-1-thio(diphosphate) (NDPαS). In some embodiments, the NDPαS is (Rp) or (Sp) diastereomer, or a mixture of (Rp) and (Sp) diastereomers.

In some embodiments, in the method for producing NQP, the method further comprises contacting a nucleoside monophosphate (NMP) with a nucleoside monophosphate kinase in presence of a phosphate donor NTP under suitable reaction conditions such that a product nucleoside diphosphate (NDP) is produced for reaction with the acetate kinase and/or 3′-O-kinase.

In some embodiments, in the method for producing NQP with a nucleoside monophosphate kinase, the NMP substrate is an unmodified or modified NMP. In some embodiments, the NMP substrate is a nucleoside 5′-O-thiophosphate (NMPαS).

In some embodiments, in the method for producing NQP using a nucleoside monophosphate kinase, the method further comprises contacting a nucleoside with a nucleoside kinase in presence of a phosphate donor NTP under reaction conditions such that a product nucleoside monophosphate (NMP) is produced.

In some embodiments, the nucleoside substrate is an unmodified or modified nucleoside. In some embodiments, the phosphate donor NTP is a nucleoside-5′-gamma thio-triphosphate (NTPγS) for producing product NMP-5′-O-thiophosphate (NMPαS) and NQPαS.

In some embodiments, a method of producing a NTP with a phosphate group at the 3′ position of the sugar moiety (NQP), comprises contacting a nucleoside with a nucleoside kinase, a nucleoside monophosphate kinase, nucleoside diphosphate kinase, and a 3′-O-kinase in presence of a phosphate donor under reaction conditions such that a product NTP with a phosphate group at the 3′ position of the sugar (NQP) is produced.

In some embodiments, a method of producing a NTP with a phosphate group at the 3′ position of the sugar (NQP), comprises (i) contacting a nucleoside with a nucleoside kinase, a nucleotide monophosphate kinase, and a nucleoside diphosphate kinase in presence of a phosphate donor under reaction conditions such that a NTP is produced; and iii) contacting the NTP produced in (i) with a 3′-O-kinase enzyme under suitable reaction conditions, such that a NTP with a phosphate group at the 3′ position of the sugar (NQP) is produced.

In some embodiments, a method of producing a NTP with a phosphate group at the 3′ position of the sugar (NQP), the method comprising (i) contacting a nucleoside with a nucleoside kinase, (ii) contacting the product of (i) with a nucleotide monophosphate kinase, (iii) contacting the product of (ii) with a nucleoside diphosphate kinase, and (iv) contacting the product of (iii) with a 3′-O-kinase enzyme, wherein (i), (ii), (iii) and (iv) are carried out in presence of a phosphate donor under reaction conditions such that a product NTP with a phosphate group at the 3′ position of the sugar (NQP) is produced.

In another aspect, the present disclosure further provides a method of producing a nucleoside triphosphate (NTP), comprising contacting a nucleoside monophosphate (NMP) with a nucleoside monophosphate kinase, and in presence of a nucleoside diphosphate kinase and a phosphate donor under reaction conditions such that NTP is produced.

In some embodiments for the method for producing NTP, the NMP substrate is an unmodified NMP or modified NMP substrate. In some embodiments, the NMP substrate comprises a nucleoside 5′-0-thiophosphate (NMPαS). In some embodiments, the product NDPαS is separated into R or S NDPαS prior to reaction with the nucleoside diphosphate kinase.

In some embodiments for the method for producing NTP, the method further comprises contacting a nucleoside with a nucleoside kinase in presence of a phosphate donor NTP under reaction conditions such that a product nucleoside monophosphate (NMP) is produced.

In some embodiments, the nucleoside is an unmodified or modified nucleoside. In some embodiments, the phosphate donor NTP has the same nucleoside structure as the nucleoside substrate. In some embodiments, the phosphate donor is NTP or NTPγS.

In some embodiments, a method of producing a nucleoside triphosphate (NTP), comprises contacting a nucleoside with a nucleoside kinase, a nucleoside monophosphate kinase, and a nucleoside diphosphate kinase in presence of a phosphate donor under reaction conditions such that NTP is produced.

In some embodiments, a method of producing a NTP comprises (i) contacting a nucleoside with a nucleoside kinase, (ii) contacting the product of (i) with a nucleotide monophosphate kinase, and (iii) contacting the product of (ii) with a nucleoside diphosphate kinase, wherein (i), (ii), and (iii) are carried out in presence of a phosphate donor under reaction conditions such that a product NTP is produced.

In the methods herein that use a nucleoside kinase, any suitable nucleoside kinase enzyme can be used. In some embodiments, the nucleoside kinase is an adenosine kinase, guanosine kinase, cytidine kinase, or uridine kinase. In some embodiments, the nucleoside kinase is an adenosine kinase. In some embodiments, the adenosine kinase is an adenosine kinase of an even-numbered SEQ ID NO. of SEQ ID NOs: 66-1204, with or without amino acid residues 1-12 (i.e., His-tag).

In the methods herein that use a nucleoside monophosphate kinase, any suitable nucleoside monophosphate kinase can be used. In some embodiments, the nucleoside monophosphate kinase is an adenylate kinase. In some embodiments, the adenylate kinase comprises an adenylate kinase of an even-numbered SEQ ID NO. of SEQ ID NOs: 1206-2504, with or without amino acid residues 1-12 (i.e., His-tag).

In the methods herein that use a nucleoside diphosphate kinase, any suitable diphosphate kinase can be used. In some embodiments, the nucleoside diphosphate kinase is an acetate kinase and the phosphate donor further includes acetyl phosphate. In some embodiments, the acetate kinase comprises an acetate kinase of an even-numbered SEQ ID NO. of SEQ ID NOs: 2506-3058, with or without amino acid residues 1-12 (i.e., His-tag).

In the methods herein that use a 3′-O-kinase, any suitable 3′-O-kinase can be used. In some embodiments, the 3′-O-kinase comprises a 3′-O-kinase of an even-numbered SEQ ID NO. of SEQ ID NOs: 3060-3370 and 3376-5126,

In some embodiments of the methods herein, the method further comprises a NTP regenerating system. In some embodiments, the NTP regenerating system comprises creatine kinase, pyruvate kinase, polyphosphate kinase, and/or R-acetate-kinase.

In some embodiments, the NTP regenerating system comprises pyruvate kinase and substrate phosphoenolpyruvate. In some embodiments, the NTP regenerating system comprises creatine kinase and substrate creatine phosphate. In some embodiments, the NTP regenerating system comprises polyphosphate kinase and substrate polyphosphate.

In some embodiments, the NTP regenerating system is R-acetate kinase and substrate acetyl phosphate. In some embodiments, the R-acetate kinase is same or different than the acetate kinase used for the conversion of NDP of NTP in the production of NQP or NTP. In some embodiments, the R-acetate kinase is an engineered acetate kinase of an even-numbered SEQ ID NO. of SEQ ID NOs: 2506-3058.

In some embodiments, where the enzymatic reaction and/or NTP regenerating system uses acetate kinase, the method further comprises pyruvate oxidase and substrate pyruvate and inorganic phosphate. In some embodiments, the method further comprises a catalase for removal of H₂O₂ generated by pyruvate oxidase reaction.

### DETAILED DESCRIPTION

Nucleoside-5′-triphosphates (NTPs) and their analogs are building blocks of polynucleotides and are important compounds in both pharmaceutical and molecular biology applications. Currently, commercially available base or sugar modified NTPs are mainly synthesized chemically. Even unmodified NTPs, though readily available, has associated high costs for preparation at industrial scale.

As an alternative to the time consuming and inefficient chemical production of NTPs the present disclosure provides an enzymatic (biocatalytic) synthesis method for the production of NQPs and NTPs, including modified NTPs. The use of efficient enzymes reduces side reactions and simplifies scale up.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used in this specification and the appended claims, the singular forms “a”, “an” and “the” include plural referents unless the context clearly indicates otherwise. Thus, for example, reference to “a polypeptide” includes more than one polypeptide. Similarly, “comprise,” “comprises,” “comprising” “include,” “includes,” and “including” are interchangeable and not intended to be limiting.

It is to be understood that where descriptions of various embodiments use the term “comprising,” those skilled in the art would understand that in some specific instances, an embodiment can be alternatively described using language “consisting essentially of” or “consisting of.”

It is to be further understood that where descriptions of various embodiments use the term “optional” or “optionally” the subsequently described event or circumstance may or may not occur, and that the description includes instances where the event or circumstance occurs and instances in which it does not.

It is to be understood that both the foregoing general description, and the following detailed description are exemplary and explanatory only and are not restrictive of this disclosure. The section headings used herein are for organizational purposes only and not to be construed as limiting the subject matter described.

### Abbreviations

The abbreviations used for the genetically encoded amino acids are conventional and are as follows:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). These abbreviations are also used interchangeably for nucleosides and nucleotides (nucleosides with one or more phosphate groups). Unless specifically delineated, the abbreviated nucleosides or nucleotides may be either ribonucleosides (or ribonucleotides) or 2′-deoxyribonucleosides (or 2′-deoxyribonucleotides). The nucleosides or nucleotides may also be modified at the 3′ position. The nucleosides or nucleotides may be specified as being either ribonucleosides (or ribonucleotides) or 2′-deoxyribonucleosides (or 2′-deoxyribonucleotides) on an individual basis or on an aggregate basis. When nucleic acid sequences are presented as a string of one-letter abbreviations, the sequences are presented in the 5′ to 3′ direction in accordance with common convention, and the phosphates are not indicated.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristoylation, ubiquitination, etc.). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

“Nucleoside kinase” refers to an enzyme that catalyzes a phosphoryl transfer from phosphate-donor molecule (e.g., a nucleotide triphosphate) to an R—OH acceptor, which is typically the 5′-hydroxyl group of the sugar moiety of the nucleoside. This process converts a nucleoside to the corresponding nucleoside monophosphate (NMP). In some embodiments, the nucleoside kinase is also referred to as a “5′-O-kinase.” In some embodiments, the nucleoside kinase catalyzes the transfer of phosphate from a phosphate-donating molecule to adenosine (A), guanosine (G), cytidine (C), thymidine, or uridine to produce adenosine monophosphate (AMP), guanosine monophosphate (GMP), cytosine monophosphate (CMP), thymidine monophosphate (TMP) or uridine monophosphate (UMP), respectively. In some embodiments, more than one nucleoside kinase is used in a reaction mixture.

“Adenosine kinase,” “AdoK” “Adk,” or “AK” refer to an enzyme that catalyzes the phosphorylation of adenosine (A or ADO) to adenosine-5′-monophosphate (AMP). In some embodiments, adenosine kinase is classified in EC 2.7.1.20. Although the primary substrate is adenosine, adenosine kinases as used herein include those that act on other nucleosides.

“Nucleoside monophosphate kinase,” “NMP kinase” or “NMPK” refers to an enzyme that catalyzes the transfer of the terminal phosphoryl group from a nucleoside triphosphate (NTP) to the phosphoryl group on a nucleoside monophosphate. This process converts a NMP to an NDP. For example, in some embodiments, the NMP kinase catalyzes the transfer of phosphate from a phosphate-donating molecule to convert AMP, GMP, CMP, TMP, or UMP to the corresponding product ADP, GDP, CDP, TDP, and UMP, respectively. In some embodiments, the phosphate donor NTP is ATP. In some embodiments, more than one NMP kinase is used in a reaction mixture.

“Adenylate kinase,” “AdyK,” or “AK” refers to an enzyme that catalyzes the interconversion of ATP, ADP, and AMP through transfer of phosphoryl groups. In some embodiments, adenylate kinase also includes enzymes capable of catalyzing the interconversion of NTP, NDP and NMP. In some embodiments, adenylate kinase are enzymes classified in EC 2.7.3.4.

“Nucleoside diphosphate kinase” or “NDP kinase” refers to an enzyme that catalyzes the transfer of a phosphoryl group of a phosphate donor compound to a nucleoside-5′-diphosphate. In some embodiments, the NDP kinase catalyzes the transfer of phosphate from a phosphate-donating compound to ADP, CDP, GDP, TDP, or UDP to produce the corresponding ATP, CTP, GTP, TTP, or UTP, respectively. In some embodiments, the phosphate donor compound is an NTP. In some embodiments, “nucleoside diphosphate kinase” includes transferase enzymes that can transfer a phosphoryl group from a phosphate donor other than NTP, e.g., acetyl phosphate, to a NDP substrate to produce NTP. In some embodiments, more than one NDP kinase is used in a reaction mixture.

“Acetate kinase (“AcK”) refers to enzymes that are capable of catalyzing the phosphorylation of nucleoside diphosphates or analogues thereof, to nucleoside triphosphates or the corresponding analogues, using acetyl phosphate or another phosphoryl group donor. Acetate kinases as used herein includes naturally occurring, wild-type enzymes or engineered enzymes. In some embodiments, acetate kinases are naturally occurring, wild-type basic metabolic enzymes found primarily in prokaryotes that catalyze the phosphorylation of acetate to acetyl-CoA in the presence of ATP. In some embodiments, acetate kinases are derived from the naturally occurring, wild-type enzymes.

“3‘—O’-kinase,” “3′O-kinase,” or “3OK” refers to an enzyme that catalyzes the phosphorylation of the 3′-OH group of the sugar moiety of a nucleoside or nucleotide to produce a nucleoside or nucleotide with a 3′-phosphate moiety. In some embodiments, the 3′-O-kinase phosphorylates the 3′-OH of a nucleoside, nucleoside monophosphate, nucleoside diphosphate, or nucleoside triphosphate.

“Pyruvate kinase” refers to an enzyme that catalyzes the conversion of ADP and phosphoenolpyruvate to ATP and pyruvate. In some embodiments, pyruvate kinase are enzymes classified in EC 2.7.1.40.

“Creatine kinase” refers to an enzyme that catalyzes the reversible interconversion of creatine:ATP to creatine phosphate:ADP.

“Polyphosphate kinase” refers to an enzyme that catalyzes the transfer of phosphate group(s) from high-energy, phosphate-donating molecules, such as polyphosphate (PolyPn), to specific substrates/molecules. Two families of polyphosphate kinases, PPKK1 and PPK2, are known. PPK1s preferentially synthesize polyphosphate from NTP and the corresponding reverse reaction, and PPK2s preferentially consume polyphosphate to phosphorylate nucleoside mono- or diphosphates, and the corresponding reverse reactions. In some embodiments, polyphosphate kinase includes enzymes classified in EC 2.7.4.1.

“Pyruvate oxidase” or “Pox” refers to an enzyme that catalyzes the reaction between pyruvate, inorganic phosphate, and oxygen to generate acetyl phosphate and H₂O₂. In some embodiments, pyruvate oxidase include enzymes classified in EC 1.2. 3.3.

“Catalase” refers to an enzyme that converts hydrogen peroxide (H₂O₂) to H₂O and O₂. Catalase can be used to remove residual hydrogen peroxide in applications where hydrogen peroxide is present or is a product in a process. In some embodiments, catalases includes enzymes classified in EC 1.11.1.6.

“Polynucleotide,” “oligonucleotide,” and “nucleic acid” are used interchangeably herein and refer to two or more nucleosides or nucleotides that are covalently linked together. The polynucleotide may be wholly comprised of ribonucleotides (i.e., RNA), wholly comprised of 2′ deoxyribonucleotides (i.e., DNA), wholly comprised of other synthetic nucleotides or comprised of mixtures of synthetic, ribo- and/or 2′ deoxyribonucleotides.

The polynucleotides may also include modified nucleotides with substitutions, including 2′ substitutions (e.g., 2′-fluoro, 2′-O-methyl, 2′-O-methoxyethyl, locked or constrained ethyl modifications, and others known to those skilled in the art). Nucleosides will be linked together via standard phosphodiester linkages or via one or more non-standard linkages, including but not limited to phosphothiolated linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino-acid sequences. Nucleobases that are modified or synthetic may comprise any known or hypothetical or future discovered modification or structure that would be recognized by one of skill in the art as a modified or synthetic nucleobase. Similarly, the terms “polynucleotide,” “oligonucleotide,” and “nucleic acid” are intended to comprise any modified or synthetic structure that is now known or discovered in the future that would be recognized by one of skill in the art as being or having the function of a “polynucleotide,” “oligonucleotide,” or “nucleic acid.” An example of a modified or synthetic structure having the function of a “polynucleotide,” “oligonucleotide,” or “nucleic acid” is PNA or peptide nucleic acid.

“Nucleobase” or “base” refers to those naturally occurring and synthetic heterocyclic moieties commonly known to those who utilize nucleic acid or polynucleotide technology to thereby generate polymers that can hybridize to polynucleotides in a sequence-specific manner. Non-limiting examples of nucleobases include, among others, adenine, cytosine, guanine, thymine, uracil, 5-propynyl-uracil, 2-thio-5-propynyl-uracil, 5-methylcytosine, pseudoisocytosine, pseudoisouridine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

“Nucleoside” refers to glycosylamines comprising a nucleobase, and a 5-carbon sugar (e.g., ribose or deoxyribose). Non-limiting examples of nucleosides include cytidine, uridine, adenosine, guanosine, thymidine, and inosine. In contrast, the term “nucleotide” refers to the glycosylamines comprising a nucleobase, a 5-carbon sugar, and one or more phosphate groups, as further described herein. In some embodiments, nucleosides can be phosphorylated by kinases to produce nucleotides.

“Nucleoside monophosphate” or “NMP” refers to glycosylamines comprising a nucleobase, a 5-carbon sugar (e.g., ribose, deoxyribose or arabinose), and a monophosphate moiety at the 5′-position of the sugar moiety. Non-limiting examples of nucleoside monophosphates include cytidine monophosphate (CMP), uridine monophosphate (UMP), adenosine monophosphate (AMP), guanosine monophosphate (GMP), thymidine monophosphate (TMP), and inosine monophosphate (IMP). In some embodiments, “nucleoside monophosphate” may refer to a non-natural (i.e., modified) nucleoside monophosphate. The terms “nucleoside” and “nucleotide” may be used interchangeably in some contexts.

“Nucleoside diphosphate” or “NDP” refers to glycosylamines comprising a nucleobase, a 5-carbon sugar (e.g., ribose, deoxyribose, or arabinose), and a diphosphate (i.e., pyrophosphate) moiety at the 5′-position of the sugar moiety. Non-limiting examples of nucleoside diphosphates include cytidine diphosphate (CDP), uridine diphosphate (UDP), adenosine diphosphate (ADP), guanosine diphosphate (GDP), thymidine diphosphate (TDP), and inosine diphosphate (IDP). In some embodiments, “nucleoside diphosphate” may refer to a non-natural (i.e., modified) nucleoside diphosphate. The terms “nucleoside” and “nucleotide” may be used interchangeably in some contexts.

“Nucleoside triphosphate” or “NTP” refers to glycosylamines comprising a nucleobase, a 5-carbon sugar (e.g., ribose, deoxyribose, or arabinose), and a triphosphate moiety at the 5′-position of the sugar moiety. In some embodiments herein, “nucleoside triphosphate” is abbreviated as “NTP”. Non-limiting examples of nucleoside triphosphates include cytidine triphosphate (CTP), uridine triphosphate (UTP), adenosine triphosphate (ATP), guanosine triphosphate (GTP), thymidine triphosphate (TTP), and inosine triphosphate (ITP). In some embodiments, “nucleoside triphosphate” may refer to a non-natural nucleoside triphosphate. The terms “nucleoside” and “nucleotide” may be used interchangeably in some contexts.

“NQP” or “3′-phosphate-nucleoside-5′-triphosphate” or “pppNp” refers to a nucleoside tetraphosphate with a 5′-triphosphate and a 3′-phosphate, e.g., pppNp. In some embodiments, NQP may include a modified NTP portion of the NQP, including modified sugar moiety, modified nucleobase, modified triphosphate, or any combinations thereof. The terms “nucleoside” and “nucleotide” may be used interchangeably in some contexts.

“Modified” in context of a nucleoside or nucleotide refers to a nucleoside or nucleotide that has been altered to a non-naturally occurring nucleoside or nucleotide. In some embodiments, modifications to nucleoside or nucleotides include modifications to the base, sugar moiety, or phosphate, or any combinations thereof. In some embodiments, the common modifications of the 2′-position of the sugar moiety with fluoro (F) or —O—CH₃ is denoted by “fN” and “mN”, respectively, where N denotes the nucleobase on the nucleoside or nucleotide. In some embodiments, the presence of a 5′-thiophosphate is denoted by “*N”.

“Locked nucleoside” or “locked nucleotide” refers to nucleoside or nucleotide, respectively, in which the ribose moiety is modified with a bridge connecting the 2′ oxygen and 4′ carbon (see, e.g., Obika et al., Tetrahedron Letters, 1997, 38(50):8735-8738; Orum et al., Current Pharmaceutical Design, 2008, 14(11):1138-1142). In some embodiments, the bridge is a methylene or ethylene bridge. In some embodiments, the ribose moiety of the locked nucleoside or locked nucleotide is in the C3′-endo (beta-D) or C2′-endo (alpha-L) conformation.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant,” “engineered,” and “non-naturally occurring” when used with reference to a cell, nucleic acid, or polypeptide, refer to a material, or a material corresponding to the natural or native form of the material, that has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The enzymes herein may be present within a cell, present in the cellular medium, or prepared in various forms, such as lysates or isolated preparations. As such, in some embodiments, the enzymes can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure enzyme composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated enzyme is a substantially pure enzyme composition.

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Conversion” refers to the enzymatic conversion of the substrate(s) to the corresponding product(s). “Percent conversion” refers to the percent of the substrate that is converted to the product within a period of time under specified conditions.

“Suitable reaction conditions” refer to those conditions in the biocatalytic reaction solution (e.g., ranges of enzyme loading, substrate loading, cofactor loading, temperature, pH, buffers, co-solvents, etc.) under which an enzyme or enzymes of the present invention is capable of converting one or more substrate compounds to a product compound. Exemplary “suitable reaction conditions” are provided in the present invention and illustrated by the Examples.

“Composition” refers to a mixture or combination of one or more substances, wherein each substance or component of the composition retains its individual properties. As used herein, a biocatalytic composition refers to a combination of one or more substances useful for biocatalysis.

“Loading”, such as in “compound loading” or “enzyme loading” or “cofactor loading” refers to the concentration or amount of a component in a reaction mixture at the start of the reaction.

“Substrate” in the context of a biocatalyst mediated process refers to the compound or molecule acted on by the biocatalyst. For example, a 3′-O-kinase biocatalyst used in the synthesis processes disclosed herein acts on a nucleoside or nucleotide substrate, e.g., NTP substrate.

“Product” in the context of a biocatalyst mediated process refers to the compound or molecule resulting from the action of the biocatalyst. For example, an exemplary product for a 3-′O-kinase biocatalyst used in a process disclosed herein is an NQP.

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁-C₆)alkyl refers to an alkyl of 1 to 6 carbon atoms).

“Alkenyl” refers to hydrocarbon groups of from 2 to 12 carbon atoms inclusively, either straight or branched containing at least one double bond but optionally containing more than one double bond.

“Alkynyl” refers to hydrocarbon groups of from 2 to 12 carbon atoms inclusively, either straight or branched containing at least one triple bond but optionally containing more than one triple bond, and additionally optionally containing one or more double bonded moieties.

“Heteroalkyl, “heteroalkenyl,” and heteroalkynyl,” refer respectively, to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to —O—, —S—, —S—O—, —NR′—, —PH—, —S(O)—, —S(O)₂—, —S(O) NR′—, —S(O)₂NR′, and the like, including combinations thereof, where each R′ is independently selected from hydrogen, alkyl, cycloalkyl, heterocycloalkyl, aryl, and heteroaryl.

“Amino” refers to the group —NH₂. Substituted amino refers to the group —NHR′, NR′R′, and NR′R′R′, where each R′ is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with one or more amino groups, including substituted amino groups.

“Aminocarbonyl” refers to —C(O)NH₂. Substituted aminocarbonyl refers to —C(O)NR′R′, where the amino group NR′R′ is as defined herein.

“Oxy” refers to a divalent group —O—, which may have various substituents to form different oxy groups, including ethers and esters.

“Alkoxy” or “alkyloxy” are used interchangeably herein to refer to the group —OR′, wherein R′ is an alkyl group, including optionally substituted alkyl groups.

“Carboxy” refers to —COOH.

“Carbonyl” refers to —C(O)—, which may have a variety of substituents to form different carbonyl groups including acids, acid halides, aldehydes, amides, esters, and ketones.

“Carboxyalkyl” refers to an alkyl in which one or more of the hydrogen atoms are replaced with one or more carboxy groups.

“Aminocarbonylalkyl” refers to an alkyl substituted with an aminocarbonyl group, as defined herein.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl group in which in which one or more of the hydrogen atoms are replaced with one or more hydroxy groups.

“Thiol” or “sulfanyl” refers to —SH. Substituted thiol or sulfanyl refers to —S—R′, where R′ is an alkyl, aryl or other suitable substituent.

“Sulfonyl” refers to —SO₂—. Substituted sulfonyl refers to —SO₂—R′, where R′ is an alkyl, aryl or other suitable substituent.

“Alkylsulfonyl” refers to —SO₂—R′, where R′ is an alkyl, which can be optionally substituted. Typical alkylsulfonyl groups include, but are not limited to, methylsulfonyl, ethylsulfonyl, n-propylsulfonyl, and the like.

“Phosphate” as used herein refers to a functional group comprised of an orthophosphate ion (phosphorous atom covalently linked to four oxygen atoms). The orthophosphate ion is commonly found with one or more hydrogen atoms or organic groups. A phosphate group or chain may be modified, as further described herein.

“Phosphorylated” as used herein refers to the addition or presence of one of more phosphoryl groups (phosphorous atom covalently linked to the three oxygen atoms).

“Thiophosphate” refers to an instance where a non-bridging oxygen in a phosphate group of a phosphodiester bond, NMP, NDP, NTP or NQP is replaced with a sulfur. In some embodiments, nucleoside 5′-thiophosphate is referred to as NMPαS or NMP-S. In some embodiments, nucleoside-5′-1-thio(diphosphate) and nucleoside-5′-1-thio(triphosphate) are referred to as NDPαS and NTPαS, respectively. In some embodiments, nucleoside-5′-2-thio(diphosphate) and nucleoside-5′-2-thio(triphosphate) are referred to as NDPβS and NTPβS, respectively. In some embodiments, nucleoside-5′-gamma-thiotriphosphate is referred to as NTPγS.

“Dithiophosphate” refers to an instance where two non-bridging oxygens in a phosphate group of a phosphodiester bond, NMP, NDP, NTP or NQP are replaced with two sulfurs.

“Optionally substituted” as used herein with respect to the foregoing chemical groups means that positions of the chemical group occupied by hydrogen can be substituted with another atom (unless otherwise specified) exemplified by, but not limited to carbon, oxygen, nitrogen, or sulfur, or a chemical group, exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; where preferred heteroatoms are oxygen, nitrogen, and sulfur. Additionally, where open valences exist on these substitute chemical groups they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further contemplated that the above substitutions can be made provided that replacing the hydrogen with the substituent does not introduce unacceptable instability to the molecules of the present invention, and is otherwise chemically reasonable. One of ordinary skill in the art would understand that with respect to any chemical group described as optionally substituted, only sterically practical and/or synthetically feasible chemical groups are meant to be included. “Optionally substituted” as used herein refers to all subsequent modifiers in a term or series of chemical groups. For example, in the term “optionally substituted arylalkyl,” the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

“Reaction” as used herein refers to a process in which one or more substances or compounds or substrates is converted into one or more different substances, compounds, or processes. In some embodiments, the reaction is described as “conversion” of a substrate to a product.

“NTP regenerating system,” “NTP regeneration system” or “NTP recycling system” refers to chemical and/or enzymatic systems for the conversion of NDP to NTP, particularly to regenerate NTP in reactions that consume NTP to produce NDP. In some embodiments, the NTP regenerating system is an enzyme catalyzed system using a phosphate donor for conversion of NDP to NTP. Exemplary enzyme catalyzed NTP regeneration system include, among others, those using pyruvate kinase, creatine kinase, polyphosphate kinase, and acetate kinase.

### Enzyme Cascades for the Synthesis of NQP

In one aspect, the present disclosure provides a process and method and corresponding enzymes for the conversion of a nucleoside diphosphate (NDP) to a NTP with a phosphate group at the 3′ position of the sugar (referred herein as a nucleoside tetraphosphate, pppNp, or NQP). The conversion reaction from NTP to NQP is illustrated in Scheme 1.

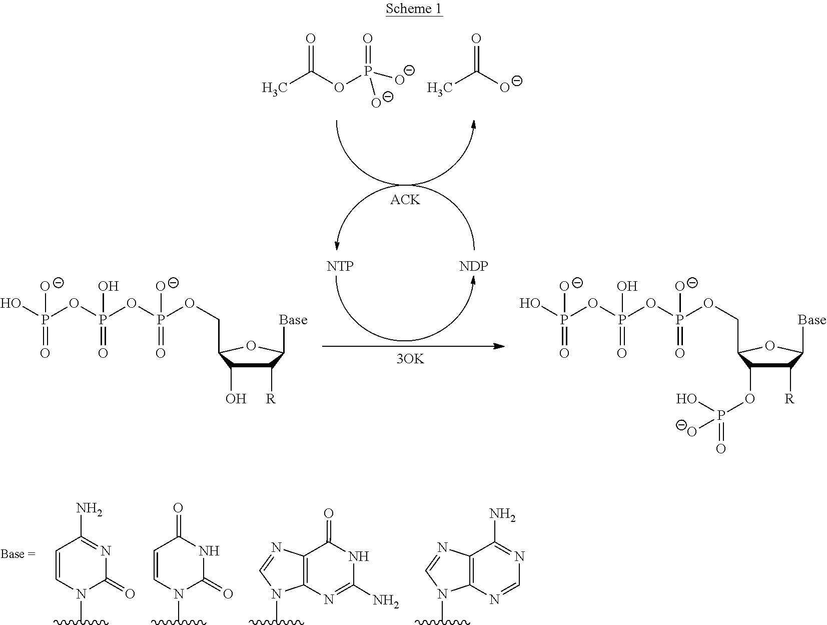

In Scheme 1, the NTP is converted by a 3′-O-kinase to a nucleoside tetraphosphate (NQP or pppNp) with the fourth phosphate group at the 3′ position of the sugar moiety. The 3′-O-kinase uses a NTP as a co-substrate and phosphate donor, simultaneously producing an NDP. A nucleoside diphosphate kinase, such as an acetate kinase (ACK), is used to recycle the NTP donor from NDP using acetyl-phosphate as a phosphate donor substrate that is converted to acetate. Use of a phosphate donor NTP that has the same nucleotide structure as the substrate NTP for the 3′-O-kinase provides for generation of the 3′-phosphorylated product without contamination from using a phosphate donor NTP with a nucleotide structure different from the substrate NTP. In some embodiments, the NTP substrate for the 3′-O-kinase reaction is generated enzymatically, such as from a nucleoside, nucleoside monophosphate, and/or nucleoside diphosphate.

Accordingly, in some embodiments, a method of producing a nucleotide triphosphate with a phosphate group at the 3′ position of the sugar moiety (NQP), comprises at least:
contacting a nucleoside diphosphate (NDP) with a nucleoside diphosphate kinase, a 3′-O-kinase, and a phosphate donor under reaction conditions such that an NTP with a phosphate group on the 3′ position of the sugar moiety (NQP) is produced.

It is to be understood that in the foregoing, the NDP can be converted to NTP and subsequently phosphorylated at the 3′-OH by the 3′-O-kinase. Alternatively, the 3′-O′-kinase can phosphorylate the 3′-OH of NDP to generate 3′-phosphate-nucleoside-5′-diphosphate (ppNp), which can then be converted to NQP by the activity of nucleoside diphosphate kinase. The product in either pathway is NQP.

In some embodiments, the NDP substrate is an unmodified NDP. In some embodiments, the unmodified NDP substrate is any naturally occurring NDP.

In some embodiments, the NDP substrate has at the 2′ position of the sugar moiety a H (e.g., deoxy) or OH (e.g., ribo).

In some embodiments, the nucleobase of the NDP substrate is, among others, adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments of the method, the NDP substrate is ADP, GDP, UDP, CDP, or TDP, and wherein the NDP has at the 2′-position of the sugar moiety an OH, thereby by resulting in corresponding product rAQP, rGQP, rUQP, rCQP, or rTQP, respectively.

In some embodiments, of the method, the NDP substrate is ADP, GDP, UDP, CDP, or TDP, and wherein the NDP has at the 2′-position of the sugar moiety a H, thereby by resulting in corresponding product dAQP, dGQP, dUQP, dCQP, or dTQP, respectively.

In some embodiments, the NDP substrate is a modified NDP, thereby resulting a modified NQP product. In some embodiments, the modified NDP comprises a modified sugar moiety, modified nucleobase, or modified phosphate, or combinations thereof.

In some embodiments, the NDP substrate comprises a modified sugar moiety. In some embodiments, the modified sugar moiety is modified at the 2′-position of the sugar moiety. In some embodiments, the modified 2′-position of the sugar moiety of the NDP substrate is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the NDP substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetalester.

In some embodiments, the modified 2′-position of the sugar moiety of the NDP substrate is halo. In some embodiments, the modified 2′-position of the sugar moiety is F (i.e., 2′-F) or Br (i.e., 2′-Br).

In some embodiments, the modified sugar moiety of the NDP substrate is a “locked” nucleotide (e.g., locked NDP). In some embodiments, the locked NDP is a locked ADP, locked GDP, locked CDP, locked TDP, or locked UDP. In some embodiments, the ribose moiety of the locked nucleotide is in the C3′-endo (beta-D-LNA) or C2′-endo (alpha-L-LNA) conformation.

In some embodiments, the modified NDP substrate comprises a modified nucleobase. In some embodiments, the modified nucleobase on the NDP substrate is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the NDP substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethy 1-2-thiouridine, 5-methylaminomethyluridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methyl-uridine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase of the NDP substrate comprises a removable tag, a cleavable linker, or a radio, a photo, and/or a chemical sensor.

In some embodiments, the modified NDP substrate comprises a modified phosphate. In some embodiments, the modified NDP substrate comprises a 5′-O-1-thio(diphosphate) (NDPαS). In some embodiments, the modified NDP substrate comprises a 5′-O-2-thio(diphosphate) (NDPβS). In some embodiments, the modified NDPαS comprises an ADPαS, GDPαS, CDPαS, TDPαS, or UDPαS. In some embodiments, the modified NDPαS comprises a rADPαS, rGDPαS, rCDPαS, rTDPαS, or rUDPαS. In some embodiments, the modified NDPαS comprises a dADPαS, dGDPαS, dCDPαS, dTDPαS, or dUDPαS.

In some embodiments, when the modified NDP substrate comprises an NDPαS, wherein the NDPαS is (Rp) or (Sp) diastereomer, or a mixture of (Rp) and (Sp) diastereomers. In some embodiments, the NDPαS is the (Rp) diastereomer. In some embodiments, the NDPαS is the (Sp) diastereomer. In some embodiments, the NDPαS is a mixture of (Rp) and (Sp) diastereomers. In some embodiments, the (Rp) or (Sp) diastereomer is provided for each of the rNTPαS and dNTPαS described herein.

In some embodiments, the method further comprises contacting a nucleoside monophosphate (NMP) with a nucleoside monophosphate kinase in presence of a phosphate donor NTP under suitable reaction conditions such that a product nucleoside diphosphate (NDP) is produced for reaction with the nucleoside diphosphate kinase and/or 3′-O-kinase.

In some embodiments, the NMP substrate is an unmodified NMP. In some embodiments, the unmodified NMP substrate is any naturally occurring NMP.

In some embodiments, the NMP substrate has at the 2′ position of the sugar moiety a H (e.g., deoxy) or OH (e.g., ribo).

In some embodiments, the nucleobase of the NMP substrate is adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments, the nucleobase of the NMP substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments of the method, the NMP substrate is AMP, GMP, UMP, CMP, or TMP, and wherein the NMP has at the 2′-position of the sugar moiety an OH, thereby by resulting in corresponding product rAQP, rGQP, rUQP, rCQP, or rTQP, respectively.

In some embodiments, of the method, the NMP substrate is AMP, GMP, UMP, CMP, or TMP, and wherein the NMP has at the 2′-position of the sugar moiety a H, thereby by resulting in corresponding product dAQP, dGQP, dUQP, dCQP, or dTQP, respectively.

In some embodiments, the NMP substrate is a modified NMP, thereby resulting a modified NQP product. In some embodiments, the modified NMP comprises a modified sugar moiety, modified nucleobase, or modified phosphate, or combinations thereof.

In some embodiments, the NMP substrate comprises a modified sugar moiety. In some embodiments, the modified sugar moiety is modified at the 2′-position of the sugar moiety. In some embodiments, the modified 2′-position of the sugar moiety of the NMP substrate is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetate ester.

In some embodiments, the modified 2′-position of the sugar moiety of the NMP substrate is halo. In some embodiments, the modified 2′-position of the sugar moiety is F (i.e., 2′-F) or Br (i.e., 2′-Br).

In some embodiments, the modified sugar moiety of the NMP substrate is a “locked” nucleotide (e.g., locked NMP). In some embodiments, the locked NMP is a locked AMP, locked GMP, locked CMP, locked TMP, or locked UMP. In some embodiments, the ribose moiety of the locked nucleotide is in the C3′-endo (beta-D) or C2′-endo (alpha-L) conformation.

In some embodiments, the modified NMP substrate comprises a modified nucleobase. In some embodiments, the modified nucleobase on the NDP substrate is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the NMP substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase of the NMP substrate comprises a removable tag, a cleavable linker, or a radio, a photo, and/or a chemical sensor.

In some embodiments, the modified NMP substrate comprises a modified phosphate. In some embodiments, the modified NMP substrate comprises a nucleoside-5′-thiophosphate (NMPαS). In some embodiments, the modified NMP substrate comprises AMPαS, GMPαS, CMPαS, TMPαS, or UMPaS. In some embodiments, the modified NMP substrate comprises rAMPαS, rGMPαS, rCMPαS, rTMPαS, or rUMPαS. In some embodiments, the modified NMP substrate comprises dAMPαS, dGMPαS, dCMPαS, dTMPαS, or dUMPαS.

In some embodiments, where the modified NMP substrate is an NMPαS, the corresponding NDPαS product of the nucleoside monophosphate kinase is isolated or purified prior to reaction with the nucleoside diphosphate kinase and/or 3′-O-kinase. In some embodiments, any resulting NDPαS diastereomeric products can be separated and used separately for subsequent reaction with the nucleoside diphosphate kinase and/or 3′-O-kinase.

In some embodiments, the phosphate donor NTP for the nucleoside monophosphate kinase comprises ATP. In some embodiments, the phosphate donor NTP for the nucleoside monophosphate kinase has the same nucleotide structure as the substrate NMP. By way of example and not limitation, where the substrate NMP is AMP, the phosphate donor is ATP. If the substrate NMP is modified at the 2′-position of the ribose, the phosphate donor NTP has the same nucleobase and modified 2′-position as the substrate NMP.

In some embodiments, the method further comprises contacting a nucleoside with a nucleoside kinase in presence of a phosphate donor NTP under reaction conditions such that a product nucleoside monophosphate (NMP) is produced for reaction with the nucleoside monophosphate kinase and/or 3′-O-kinase.

In some embodiments, the nucleoside substrate is an unmodified nucleoside. In some embodiments, the unmodified nucleoside substrate is any naturally occurring nucleoside.

In some embodiments, the nucleoside substrate has at the 2′-position of the sugar moiety a H (e.g., deoxy) or OH (e.g., ribo).

In some embodiments, the nucleobase of the nucleoside substrate is adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments of the method, the nucleoside substrate is adenosine (A), guanosine (U), uridine (U), cytidine (C), or thymidine (T), and wherein the nucleoside has at the 2′-position of the sugar moiety an OH, thereby by resulting in corresponding product rAQP, rGQP, rUQP, rCQP, or rTQP, respectively.

In some embodiments of the method, the nucleoside substrate is adenosine (A), guanosine (U), uridine (U), cytidine (C), or thymidine (T), and wherein the nucleoside has at the 2′-position of the sugar moiety a H, thereby by resulting in corresponding product dAQP, dGQP, dUQP, dCQP, or dTQP, respectively.

In some embodiments, the nucleoside substrate is a modified nucleoside, thereby resulting in a modified NQP product. In some embodiments, the modified nucleoside is modified on the sugar moiety or nucleobase, or combinations thereof.

In some embodiments, the nucleoside substrate comprises a modified sugar moiety. In some embodiments, the modified sugar moiety is modified at the 2′-position of the sugar moiety. In some embodiments, the modified 2′-position of the sugar moiety of the nucleoside substrate is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetate ester.

In some embodiments, the modified 2′-position of the sugar moiety of the nucleoside substrate is halo. In some embodiments, the modified 2′-position of the sugar moiety is F (i.e., 2′-F) or Br (i.e., 2′-Br).

In some embodiments, the modified sugar moiety of the nucleoside substrate is a “locked” nucleotide (e.g., locked nucleoside). In some embodiments, the locked nucleoside is a locked adenosine, locked guanosine, locked cytidine, locked thymidine, or locked uridine. In some embodiments, the ribose moiety of the locked nucleoside is in the C3′-endo (beta-D-LNA) or C2′-endo (alpha-L-LNA) conformation.

In some embodiments, the modified nucleoside substrate comprises a modified nucleobase. In some embodiments, the modified nucleobase on the nucleoside substrate is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the nucleoside substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase of the nucleoside substrate comprises a removable tag, a cleavable linker, or a radio, a photo, and/or a chemical sensor.

In some embodiments, the phosphate donor NTP for the nucleoside kinase comprises ATP. In some embodiments, the phosphate donor NTP for the nucleoside kinase has the same nucleotide structure as the substrate nucleoside. By way of example and not limitation, where the substrate nucleoside is adenosine, the phosphate donor is ATP. If the substrate nucleoside is modified at the 2′-position of the ribose, the phosphate donor NTP has the same nucleobase and modified 2′-position as the substrate nucleoside.

In some embodiments, the phosphate donor NTP is a nucleoside-5′-gamma thiotriphosphate (NTPγS) for producing products NMP-5′-O-thiophosphate (NMP-S) and NQPαS. In some embodiments, the phosphate donor NTPγS is rATPγS or dATPγS.

In some embodiments, a method of producing a NTP with a phosphate group at the 3′ position of the sugar moiety (NQP), comprises contacting a nucleoside with a nucleoside kinase, a nucleoside monophosphate kinase, nucleoside diphosphate kinase, and a 3′-O-kinase in presence of a phosphate donor under reaction conditions such that a product NTP with a phosphate group at the 3′ position of the sugar (NQP) is produced.

In some embodiments, a method of producing a NTP with a phosphate group at the 3′ position of the sugar moiety (NQP) comprises (i) contacting a nucleoside with a nucleoside kinase, a nucleotide monophosphate kinase, and a nucleoside diphosphate kinase in presence of a phosphate donor under reaction conditions such that a NTP is produced; and ii) contacting the NTP produced in (i) with a 3′-O-kinase under suitable reaction conditions, such that a NTP with a phosphate group at the 3′ position of the sugar moiety (NQP) is produced.

In some embodiments, a method of producing a NTP with a phosphate group at the 3′ position of the sugar moiety (NQP) comprises (i) contacting a nucleoside with a nucleoside kinase, (ii) contacting the product of (i) with a nucleotide monophosphate kinase, (iii) contacting the product of (ii) with a nucleoside diphosphate kinase, and (iv) contacting the product of (iii) with a 3′-O-kinase enzyme, wherein (i), (ii), (iii) and (iv) are carried out in presence of a phosphate donor under reaction conditions such that a product NTP with a phosphate group at the 3′ position of the sugar moiety (NQP) is produced.

In some embodiments, a method of producing a NTP with a phosphate group at the 3′ position of the sugar or NQP (pppNp), comprises i) contacting the 3′-O-kinase with a nucleoside under suitable reaction conditions, such that a nucleoside with a phosphate at the 3′-position of the sugar is produced (Np); (ii) contacting the product of (i), i.e., nucleoside with nucleoside with a phosphate at the 3′ position of the sugar (Np) with a nucleoside kinase (5-′O-kinase), a nucleotide monophosphate kinase, and a nucleoside diphosphate kinase (e.g., an acetate kinase) under suitable reaction conditions, such that a NTP with a phosphate group at the 3′ position of the sugar or NQP (pppNp) is produced.

In some embodiments of the foregoing, the nucleoside, nucleoside monophosphate, nucleoside diphosphate, and nucleoside triphosphate for each of the enzymatic reactions are as described above.

In some embodiments, the nucleoside substrate is an unmodified nucleoside.

In some embodiments, the nucleoside has at the 2′-position of the sugar moiety a H or OH.

In some embodiments, the nucleobase of the nucleoside substrate is adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments, the nucleoside substrate is adenosine (A), guanosine (G), uridine (U), cytidine (C), or thymidine (T), and wherein the nucleoside has at the 2′-position of the sugar moiety an OH, thereby by resulting in corresponding product rAQP, rGQP, rUQP, rCQP, or rTQP, respectively.

In some embodiments, the nucleoside substrate is adenosine (A), guanosine (G), uridine (U), cytidine (C), or thymidine (T), and wherein the nucleoside has at the 2′-position of the sugar moiety a H, thereby by resulting in corresponding product dAQP, dGQP, dUQP, dCQP, or dTQP, respectively.

In some embodiments, the nucleoside substrate is a modified nucleoside. In some embodiments, the nucleoside has a modified sugar moiety or modified nucleobase, or combination thereof.

In some embodiments, the modified nucleoside substrate has a modified 2′-position of the sugar moiety. In some embodiments, the 2′-position of the sugar moiety is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetate ester.

In some embodiments, the 2′-position of the sugar moiety is halo. In some embodiments, the 2′-position of the sugar moiety is F (i.e., 2′-F) or Br (i.e., 2′-Br).

In some embodiments, the modified sugar moiety is a locked nucleoside. In some embodiments, the locked nucleoside is a locked adenosine, locked guanosine, locked cytidine, locked thymidine, or locked uridine. In some embodiments, the ribose moiety of the locked nucleoside is in the C3′-endo (beta-D) or C2′-endo (alpha-L) conformation.

In some embodiments, the modified nucleoside substrate has a modified nucleobase. In some embodiments, the modified nucleobase of the nucleoside substrate is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the nucleoside substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase of the nucleoside substrate comprises a noncanonical nucleobase, a removable tag, a cleavable linker, or a radio, a photo, and/or a chemical sensor.

In some embodiments of the enzymatic method, the phosphate donor comprises a phosphate donor NTP. In some embodiments, as further described below, where the nucleoside diphosphate kinase comprises an acetate kinase, the phosphate donor further includes acetyl phosphate.

In some embodiments, the phosphate donor NTP for the enzymatic method for NQP synthesis comprises ATP. In some embodiments, the phosphate donor NTP has the same nucleotide structure as the nucleoside substrate. In some embodiments, the phosphate donor NTP is a nucleoside-5′-1-thio(triphosphate) (NTPαS)

In some embodiments, the phosphate donor NTP is a nucleoside-5′-gamma thio-triphosphate (NTPγS) for producing products NMP-5′-O-thiophosphate (NMP-S) and NQPαS. In some embodiments, the phosphate donor NTPγS is rATPγS or dATPγS.

In some embodiments, each of the enzymatic steps for production of NQP can be carried out separately. In some embodiments, the products of each step can be isolated prior to reaction with the next enzymatic step. In some embodiments, the products are not isolated following each enzymatic step.

In some embodiments, the enzymatic conversion of a nucleoside substrate to the corresponding NTP can be carried in a first reaction, and the conversion of the NTP to the corresponding NQP with 3′-O-kinase carried out in a second reaction. In some embodiments herein, this is referred to as a “two-pot” method or process.

In some embodiments, the enzymatic conversion of a nucleotide substrate to the corresponding NQP can be carried out in a single reaction, e.g., nucleoside kinase, nucleoside monophosphate kinase, nucleoside diphosphate kinase, and 3′-O-kinase, nucleoside substrate, phosphate donor, and any other accessory enzymes, e.g., NTP regenerating system, are in a single reaction. In some embodiments, this is referred to as a “one-pot” method or process.

In some embodiments, the present disclosure provides an exemplary one-pot method for conversion of nucleosides to NQPs, as depicted in Scheme 2.

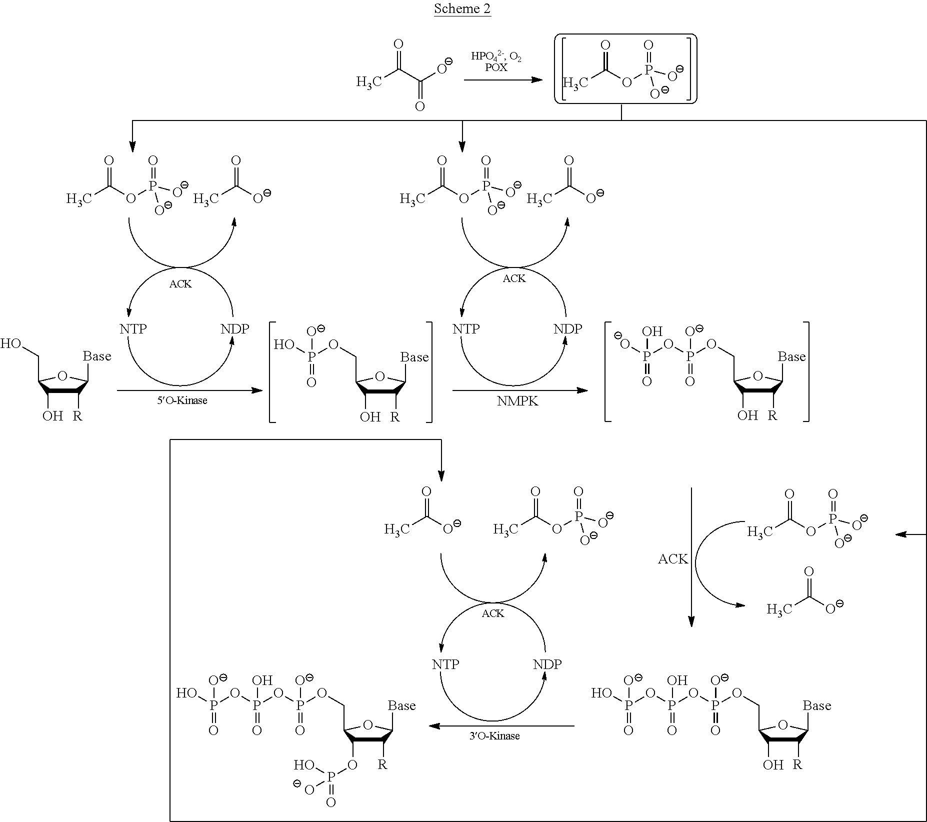

In some embodiments, the one-pot method for conversion of nucleosides to NQPs occurs in more than one step. In some embodiments, the one-pot method for conversion of nucleosides to NQPs occurs in two steps. In some embodiments, the first step comprises conversion of a nucleoside to an NTP (as exemplified in Scheme 3, below), and the second step comprises conversion of the NTP to an NQP (as exemplified in Scheme 1, above).

In some other embodiments, the first step comprises conversion of a nucleoside to a nucleoside with a phosphate at the 3′ position, and the second step comprises conversion of the nucleoside with a phosphate at the 3′ position to an NQP.

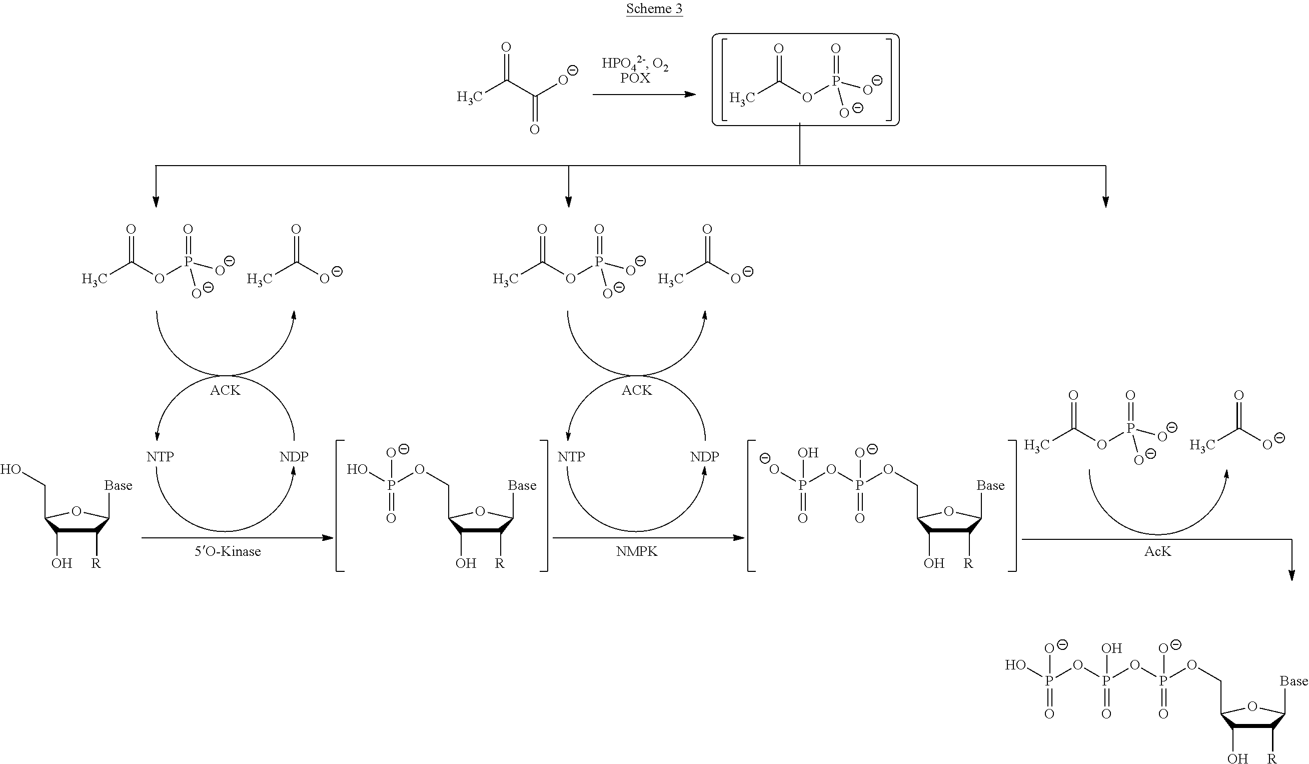

The first step of the two step one-pot method for conversion of nucleosides to NQPs comprises use of a nucleoside kinase (5′-O-kinase), a nucleoside monophosphate kinase, and an acetate kinase (AcK) to sequentially add three phosphates (or modified phosphates or phosphate substitutes) to a nucleoside to generate an NTP.

In some embodiments, as exemplified in Scheme 3, the nucleoside is first converted by a nucleoside kinase (5′-O-kinase) to a NMP by addition of a phosphate group to the 5′-OH position of the sugar moiety. After conversion of the nucleoside to a NMP by the nucleoside kinase, the NMP is converted to an NDP by a nucleoside monophosphate kinase (NMPK). Then, the NDP is converted to an NTP by a nucleoside diphosphate kinase (e.g., acetate kinase: AcK).

In some embodiments, with reference to Scheme 3, the 2′-R group can be —H, —OH, —OCH₃, —F, —OCH₂CH₂OCH₃, 2′-O—C(O)R′ (where R′ is an alkyl or aryl), or another atom or chemical group. Additionally, the sugar moiety may have other modifications at other positions. In some embodiments, the nucleobase may be an adenine, guanine, uridine, thymine, or cytosine, or another nucleobase known to those skilled in the art. Although not depicted in Scheme 3, the nucleoside may also have modifications of the nucleobase or of the 5′ phosphate chain. Any of these modifications may be present in any combination in any of the substrates or products depicted in Scheme 3 or may be added after conversion to the NTP product or may be added during or after the second step of the conversion depicted in Scheme 1.

In some embodiments, as depicted in Scheme 3, the nucleoside kinase (5′-O-kinase) and nucleoside monophosphate kinase use a NTP as a co-substrate and phosphate donor, simultaneously producing an NDP. In addition to catalyzing the conversion of the NDP to NTP, the acetate kinase is used to recycle the NTP donor from NDP using acetyl phosphate as a donor substrate that is converted to acetate, as further described herein. However, this is only one embodiment of the present invention, which is not intended to be so limited. The phosphate used by the nucleoside kinase (5′-O-kinase) and nucleoside monophosphate kinase may be sourced from any suitable molecule, with or without a recycling enzyme, as is known by those of skill in the art.

In certain embodiments where the nucleoside kinase (5-′O-kinase) and nucleoside monophosphate kinase enzymes are coupled with an acetate kinase recycling enzyme, the acetate kinase enzyme may be further coupled with a pyruvate oxidase enzyme (POX) to generate acetyl phosphate from pyruvate, as depicted in Scheme 3, as describe below. In some embodiments, the pyruvate oxidase enzyme transiently generates acetyl phosphate from pyruvate, atmospheric oxygen, and potassium phosphate buffer. These embodiments have the advantage of generating an unstable, moisture sensitive, and expensive substrate (acetyl phosphate) from stable, readily available, and inexpensive reagents (pyruvate, atmospheric oxygen, and potassium phosphate buffer).

The second step of the two step one-pot method for conversion of nucleosides to NQPs comprises use of a 3-′O-kinase to convert the natural or modified NTP generated in step one to the NQP product. The second step of the two step one-pot method conversion of nucleosides to NQPs, is described above and depicted in Scheme 1.

Thus, the two step one-pot method for conversion of nucleosides to NQPs comprises i) a first step comprising providing a nucleoside kinase (5′-O-kinase) enzyme, a nucleoside monophosphate enzyme, and an acetate kinase enzyme under suitable reaction conditions for conversion of a natural or modified nucleoside to a natural or modified NTP, optionally including an AcK recycling enzyme and/or POX enzyme and/or other suitable recycling enzymes; ii) a second step comprising providing a 3′-O-kinase enzyme under suitable reaction conditions for conversion of a natural or modified NTP to a natural or modified NQP, optionally including an ACK recycling enzyme and/or POX enzyme or other suitable recycling enzymes; and iii) optionally, comprising providing one or more additional enzymes or catalysts to generate one or more modifications to a nucleoside, NMP, NDP, NTP, or NQP as part of the one-pot method.

As described above, in some embodiments, the one-pot method for conversion of nucleosides to NQPs comprises i) a step comprising providing a nucleoside kinase (5′-O-kinase) enzyme, a nucleoside monophosphate enzyme, an acetate kinase enzyme, and a 3′-O-kinase enzyme under suitable reaction conditions for conversion of a natural or modified nucleoside to a natural or modified NQP, optionally including an AcK enzyme and/or POX enzyme and/or other suitable recycling enzymes; and ii) optionally, comprising providing one or more additional enzymes or catalysts to generate one or more modifications to a nucleoside, NMP, NDP, NTP, or NQP as part of the one-pot method.

In any of the above embodiments, the nucleosides, NMPs, NDPs, NTPs, or NQPs, and other byproduct species may be natural or may comprise one or more modifications, as described herein. In any of the above embodiments, the nucleosides, NMPs, NDPs, NTPs, and NQPs, and other byproduct species may comprise ribonucleosides, deoxyribonucleosides, dideoxynucleosides, or modified nucleosides. In any of the above embodiments, the nucleosides, NMPs, NDPs, NTPs, and NQPs, and other byproduct species may comprise one or more modifications to the sugar. In any of the above embodiments, the nucleosides, NMPs, NDPs, NTPs, and NQPs, and other byproduct species may comprise one or more modifications to the nucleobase. In any of the above embodiments, the NMPs, NDPs, NTPs, and NQPs, and other byproduct species may comprise an α-thiophosphate or dithiophosphate or other modification to the 5′ phosphate chain. In some embodiments, any of these modifications may be present in any combination in the 3′-O-kinase substrate or may be added after or during conversion to the 3′-O-kinase product.

In some embodiments, the 2′-R group of the sugar comprises H, OH, OCH₃, OCH₂CH₂OCH₃, F, 2′-O—C(O)R′ (where R′ is any alkyl or aryl), or another atom or chemical group. In some embodiments, the sugar may have other modifications at other positions, such as locked nucleotides or constrained ethyl nucleotides, as is known in the art. In some embodiments, the 3′-phosphate group of the NQP may act as a removable blocking group or protecting group that may be selectively unblocked or removed to allow further modifications, reactions, or incorporation of the NQP into a growing oligonucleotide chain during template-dependent or template-independent oligonucleotide synthesis.

In some embodiments, as described elsewhere herein, the nucleobase may be a uridine, thymine, cytosine, adenine, guanine or another nucleobase known to those skilled in the art. In some embodiments, the nucleobase of the nucleoside, NMP, NTP, NDP, NTP, and NQP, or byproduct species may have modifications. Various modified nucleobases are known to those skilled in the art, including but not limited to the following: 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethy1-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methyl-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the nucleobase modification is a removable tag, a cleavable linker, or a radio, photo, or chemical sensor. In some embodiments, the nucleobase modification is a functional element that may be used for isolation, purification, detection, protection, prevention of hydrolysis or degradation, chemical transformation, or to enable further or sequential modifications.

In some embodiments, the NMP, NTP, NDP, NTP, and NQP, or byproduct species comprises one or more modifications to the 5′ phosphate chain. The 5′ phosphate chain may comprise one, two, or three phosphates or no phosphates may be present. The 5′ phosphate chain may also comprise one or more phosphate groups with modifications (e.g. an α-thiophosphate or dithiophosphate).

In some embodiments, the present disclosure provides a method of producing a NTP with a phosphate group at the 3′-position of the sugar or NQP, the method comprising (i) providing a nucleoside kinase (5′-O-kinase) enzyme, a nucleotide monophosphate kinase enzyme, an acetate kinase enzyme, and a 3′-O-kinase enzyme, and (ii) contacting the nucleoside kinase (5′-O-kinase) enzyme, the nucleotide monophosphate kinase enzyme, the acetate kinase enzyme, and the 3′-O-kinase enzyme with a nucleoside under suitable reaction conditions, such that a NTP with a phosphate group at the 3′ position of the sugar or NQP is produced.

In certain embodiments, the method comprises (i) providing a nucleoside kinase (5′-O-kinase) enzyme, a nucleotide monophosphate kinase enzyme, an acetate kinase enzyme, and a 3-′O-kinase enzyme and (ii) contacting the nucleoside kinase (5-′O-kinase) enzyme, the nucleotide monophosphate kinase enzyme, the acetate kinase enzyme and the 3′-O-kinase enzyme with a nucleoside with one or modifications under suitable reaction conditions, such that a NTP with a phosphate group at the 3′ position of the sugar or NQP with one or more modifications is produced.

In any of the above embodiments, the method may further comprise providing a source of phosphate, and, optionally, one or more phosphate recycling enzymes, including but not limited to pyruvate oxidase and/or acetate kinase. In any of the above embodiments, the method may further comprise providing more than one 3′-O-kinase in step (i). In any of the above embodiments, the method may further comprise a phosphate donor NTP that is the same type of NTP as the substrate NTP. In any of the above embodiments, the method may further comprise a phosphate donor NTP that is a different type of NTP than the substrate NTP.

In some embodiments, the present disclosure provides a method of producing a NTP with a phosphate group at the 3′ position of the sugar or NQP, the method comprising (i) providing a nucleoside kinase (5′-O-kinase) enzyme, a nucleotide monophosphate kinase enzyme, and an acetate kinase enzyme; (ii) contacting the nucleoside kinase (5′-O-kinase) enzyme, the nucleotide monophosphate kinase enzyme, and the acetate kinase enzyme with a nucleoside under suitable reaction conditions, such that a NTP is produced; iii) providing a 3′-O-kinase enzyme; and (iv) contacting the 3′-O-kinase enzyme with the NTP under suitable reaction conditions, such that a NTP with a phosphate group at the 3′ position of the sugar or NQP is produced.

In certain embodiments, the method comprises (i) providing a nucleoside kinase (5′-O-kinase) enzyme, a nucleotide monophosphate kinase enzyme, and an acetate kinase enzyme; (ii) contacting the nucleoside kinase (5′-O-kinase) enzyme, the nucleotide monophosphate kinase enzyme, and the acetate kinase enzyme with a nucleoside with one or more modifications under suitable reaction conditions, such that a NTP with one or more modifications is produced; iii) providing a 3′-O-kinase enzyme; and (iv) contacting the 3′-O-kinase enzyme with the NTP with one or more modifications under suitable reaction conditions, such that a NTP with a phosphate group at the 3′ position of the sugar or NQP with one or more modifications is produced.

### Enzyme Cascades for NTP Synthesis

In another aspect, the present disclosure provides a method or process of producing nucleotide triphosphate (NTP) enzymatically, particularly in an enzyme cascade reaction.

In some embodiments, a method of producing a nucleoside triphosphate (NTP) comprises contacting a nucleoside monophosphate (NMP) with a nucleoside monophosphate kinase, a nucleoside diphosphate kinase and a phosphate donor under reaction conditions such that NTP is produced.

In some embodiments, the NMP is an unmodified NMP substrate.

In some embodiments, the NMP has at the 2′-position of the sugar moiety a H or OH.

In some embodiments, the nucleobase of the NMP substrate is adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments, the NMP substrate is AMP, GMP, UMP, CMP, or TMP, and wherein the NMP has at the 2′-position of the sugar moiety an OH, thereby by resulting in corresponding product rATP, rGTP, rUTP, rCTP, or rTTP, respectively.

In some embodiments, the NMP substrate is AMP, GMP, UMP, CMP, or TMP, and wherein the NMP has at the 2′-position of the sugar moiety a H, thereby by resulting in corresponding product dATP, dGTP, dUTP, dCTP, or dTTP, respectively.

In some embodiments, the NMP substrate is a modified NMP. In some embodiments, the modified NMP comprises a modified sugar moiety, nucleobase, phosphate, or any combinations thereof.

In some embodiments, the modified NMP substrate has a modified 2′-position or 3′-position of the sugar moiety.

In some embodiments, the modified NMP substrate has a modified 2′-position of the sugar moiety. In some embodiments, the 2′-position of the sugar moiety is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the NMP substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetate ester.

In some embodiments, the 2′-position of the sugar moiety is halo. In some embodiments, 2′-position of the sugar moiety is F (i.e., 2′-F) or Br (i.e., 2′-Br).

In some embodiments, the modified NMP substrate is a locked NMP. In some embodiments, the locked NMP is a locked AMP, locked GMP, locked CMP, locked TMP, or locked UMP. In some embodiments, the ribose moiety of the locked nucleoside or locked nucleotide is in the C3′-endo (beta-D) or C2′-endo (alpha-L) conformation.

In some embodiments, the modified NMP substrate has a modified 3′-position of the sugar moiety. In some embodiments, the 3′-position of the sugar moiety is modified with a blocking group, preferably a reversible blocking group. In some embodiments, the blocking group is a formate, benzoylformate, acetate, propionate, isobutyrate, aminoxy (—ONH₂), O-methyl, O-methoxymethyl, O-methylthiomethyl, O-benzyloxymethyl, O-allyl, O-propargyl, O-(2-nitrobenzyl), O-azidomethyl (O—CH₂N₃), O-tert-butyldithiomethyl, phosphate, disphosphate, or triphosphate. Reversible 3′-blocked nucleoside/nucleotides are described in Chen et al., Genomics, Proteomics & Bioinformatics, 2013, 11(1):34-40, Metzker et al., Nucleic Acids Res., 1994, 22 (20):4259-4267; and patent publications U.S. Pat. Nos. 5,763,594, 9,650,406, US20200216891; WO2004/018497; and WO 2014/139596; all of which are incorporated by reference).

In some embodiments, the modified NMP has a modified nucleobase. In some embodiments, the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, as described elsewhere herein, the nucleobase of the NMP substrate is 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase of the modified NMP substrate comprises a noncanonical nucleobase, a removable tag, a cleavable linker, or a radio, a photo, and/or a chemical sensor.

In some embodiments, the modified NMP substrate has a modified phosphate. In some embodiments, the modified NMP substrate is a nucleoside-5′-O-thiophosphate (NMPαS). In some embodiments, where the modified NMP substrate is NMPαS, the corresponding product NDPαS is separated into (Rp) or (Sp) diastereomers prior to reaction with the nucleoside diphosphate kinase.

In some embodiments, the phosphate donor is a phosphate donor NTP. In some embodiments, the phosphate donor NTP has the same nucleotide structure as the NMP substrate. In some embodiments, the phosphate donor NTP is ATP, GTP, CTP, TTP, or UTP. In some embodiments, the phosphate donor also includes acetyl phosphate where the nucleoside diphosphate kinase is an acetate kinase, as described below.

In some embodiments of the method for converting an NMP substrate to product NTP, the method further comprises contacting a nucleoside with a nucleoside kinase in presence of a phosphate donor NTP under reaction conditions such that a product nucleoside monophosphate (NMP) is produced as substrate for the nucleoside monophosphate kinase.

In some embodiments, the nucleoside substrate is an unmodified nucleoside.

In some embodiments, the nucleoside has at the 2′-position of the sugar moiety a H or OH.

In some embodiments, the nucleobase of the nucleoside substrate is adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments, the nucleoside substrate is adenosine (A), guanosine (G), uridine (U), cytidine (C), or thymidine (T), and wherein the nucleoside has at the 2′-position of the sugar moiety an OH, thereby by resulting in corresponding product rATP, rGTP, rUTP, rCTP, or rTTP, respectively.

In some embodiments, the nucleoside substrate is adenosine (A), guanosine (G), uridine (U), cytidine (C), or thymidine (T), and wherein the nucleoside has at the 2′-position of the sugar moiety a H, thereby by resulting in corresponding product dATP, dGTP, dUTP, dCTP, or dTTP, respectively.

In some embodiments, the nucleoside substrate is a modified nucleoside. In some embodiments, the nucleoside has a modified sugar moiety, modified nucleobase, or a combination of modified sugar moiety and modified nucleobase.

In some embodiments, the modified nucleoside substrate has a modified 2′-position or 3′-position of the sugar moiety.

In some embodiments, the modified nucleoside has a modified 2′-position of the sugar moiety. In some embodiments, the 2′-position of the sugar moiety is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)— alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetate ester.

In some embodiments, the 2′-position of the sugar moiety is halo. In some embodiments, the halo is F (i.e., 2′-F) or Br (i.e., 2′-Br).

In some embodiments, the modified nucleoside substrate is a locked nucleoside. In some embodiments, the locked nucleoside is a locked adenosine, locked guanosine, locked cytidine, locked thymidine, or locked uridine. In some embodiments, the ribose moiety of the locked nucleoside is in the C3′-endo (beta-D) or C2′-endo (alpha-L) conformation.

In some embodiments, the 3′-position of the sugar moiety of the nucleoside substrate is modified with a blocking group, preferably a reversible blocking group. In some embodiments, the 3′-position blocking group is formate, benzoylformate, acetate, propionate, isobutyrate, aminoxy (—ONH₂), O-methyl, O-methoxymethyl, O-methylthiomethyl, O-benzyloxymethyl, O-allyl, O-propargyl, O-(2-nitrobenzyl), O-azidomethyl (O—CH₂N₃), O-tert-butyldithiomethyl, phosphate, diphosphate, diphosphate, or triphosphate. In some embodiments, the modified sugar moiety is a 3′-O-phosphate.

In some embodiments, the modified nucleoside substrate has a modified nucleobase. In some embodiments, the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the nucleoside substrate is 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase on the nucleoside substrate comprises a noncanonical nucleobase, a removable tag, a cleavable linker, or a radio, a photo, and/or a chemical sensor.

In some embodiments, the phosphate donor for the nucleoside kinase is a phosphate donor NTP. In some embodiments, the phosphate donor NTP has the same nucleoside structure as the nucleoside substrate. In some embodiments, the phosphate donor NTP is rATP or dATP. In some embodiments, the phosphate donor NTP is ATP, GTP, CTP, TTP, or UTP. In some embodiments, the phosphate donor NTP is NTPγS.

In some embodiments, a method of producing a nucleoside triphosphate (NTP) comprises contacting a nucleoside with a nucleoside kinase, a nucleoside monophosphate kinase, and a nucleoside diphosphate kinase in presence of a phosphate donor under reaction conditions such that NTP is produced.

In some embodiments, a method of producing a NTP, comprising (i) contacting a nucleoside with a nucleoside kinase, (ii) contacting the product of (i) with a nucleotide monophosphate kinase, and (iii) contacting the product of (ii) with a nucleoside diphosphate kinase, wherein (i), (ii), and (iii) are carried out in presence of a phosphate donor under reaction conditions such that a product NTP is produced.

In some embodiments of the forgoing methods, the nucleoside substrate is an unmodified nucleoside.

In some embodiments, the nucleoside has at the 2′-position of the sugar moiety a H or OH.

In some embodiments, the nucleobase of the nucleoside substrate is adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, or 7-methylguanosine (m⁷G).

In some embodiments, the nucleoside substrate is adenosine (A), guanosine (G), uridine (U), cytidine (C), or thymidine (T), and wherein the nucleoside has at the 2′-position of the sugar moiety an OH, thereby by resulting in corresponding product rATP, rGTP, rUTP, rCTP, or rTTP, respectively.

In some embodiments, the nucleoside substrate is adenosine (A), guanosine (G), uridine (U), cytidine (C), or thymidine (T), and wherein the nucleoside has at the 2′-position of the sugar moiety a H, thereby by resulting in corresponding product dATP, dGTP, dUTP, dCTP, or dTTP, respectively.

In some embodiments of the foregoing methods, the nucleoside substrate is a modified nucleoside. In some embodiments, the modified nucleoside has a modified sugar moiety or modified nucleobase, or a combination thereof.

In some embodiments, the modified nucleoside has a modified 2′-position or 3′-position of the sugar moiety.

In some embodiments, the modified nucleoside has a modified 2′-position of the sugar moiety. In some embodiments, the 2′-position of the sugar moiety is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetalester.

In some embodiments, the 2′-position of the sugar moiety is halo. In some embodiments, the 2′-position of the sugar moiety is halo is F (i.e., 2′-F) or Br (i.e., 2′-Br).

In some embodiments, the modified nucleoside substrate is a locked nucleoside. In some embodiments, the locked nucleoside is a locked adenosine, locked guanosine, locked cytidine, locked thymidine, or locked uridine. In some embodiments, the ribose moiety of the locked nucleoside is in the C3′-endo (beta-D) or C2′-endo (alpha-L) conformation.

In some embodiments, the 3′-position of the sugar moiety is modified with a blocking group, preferably a reversible blocking group. In some embodiments, the 3′-position blocking group is formate, benzoylformate, acetate, propionate, isobutyrate, aminoxy (—ONH₂), O-methyl, O-methoxymethyl, O-methylthiomethyl, O-benzyloxymethyl, O-allyl, O-propargyl, O-(2-nitrobenzyl), O-azidomethyl (O—CH₂N₃), O-tert-butyldithiomethyl, phosphate, or triphosphate. In some embodiments, the modified sugar moiety is a 3′-O-phosphate.

In some embodiments, the modified nucleoside has a modified nucleobase. In some embodiments, the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the nucleoside substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-propynyl-uracil, 2-thio-5-propynyl-uracil, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethy luridine, 5-methylcarbon ylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethy 1-adenosine, aminoally 1-adenosine, 5′-methy 1-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments, the modified nucleobase comprises a noncanonical nucleobase, a removable tag, a cleavable linker, or a radio, a photo, and/or a chemical sensor

In some embodiments, the phosphate donor is a phosphate donor NTP. In some embodiments, the phosphate donor NTP has the same nucleoside structure as the nucleoside substrate. In some embodiments, the phosphate donor NTP is rATP or dATP. In some embodiments, the phosphate donor NTP is ATP, GTP, CTP, TMP, or UTP. In some embodiments, the phosphate donor is a phosphate donor NTPγS. As further discussed below, where the nucleoside diphosphate kinase is an acetate kinase, the phosphor donor further includes acetyl phosphate.

### NTP Regenerating Systems

In some embodiments, the methods of preparing NTP and/or NQP further includes an NTP recycling or NTP regenerating system. As used herein an NTP recycling system refers to at least regeneration of NTP from NDP formed in the enzyme and cascade reaction(s). In some embodiments, the NTP regenerating system is an enzyme-mediated NTP regeneration system.

In some embodiments, the NTP recycling system includes at least an enzyme for the conversion of NDP to NTP, and a phosphate donor. In some embodiments, the NTP recycling system includes, among others, at least an acetate kinase, adenylate kinase, pyruvate kinase, creatine kinase, or polyphosphate kinase (see, e.g., Endo et al., Adv. Synth. Catal., 2002, 343:521-526; Andexer et al., Chem Bio Chem., 2015, 16:380-386). In some embodiments, the NTP regenerating system selected is capable regenerating natural and/or modified NTPs from the respective NDPs (see, e.g., Ishige et al., Biosci. Biotechnol. Biochem., 2001, 65 1736-1740.2001; Zou et al., Appl. Microbiol. Biotechnol., 97:9389-9395).

In the NTP regeneration system, the phosphate donor or phosphate donor for the conversion of NDP to NTP is selected based on the NTP regenerating enzyme employed. By way of example and not limitation, if acetate kinase enzyme is used for conversion of NDP to NTP, the phosphate donor is acetyl-phosphate. If pyruvate kinase is used for the conversion of NDP to NTP, the phosphate donor is phosphoenolpyruvate. If creatine kinase is used for the conversion of NDP to NTP, the phosphate donor is creatine phosphate. If polyphosphate kinase is used for the conversion of NDP to NTP, the phosphate donor is inorganic polyphosphate.

In some embodiments, where the NTP recycling system uses acetate kinase for the conversion of NDP to NTP, the acetate kinase can be the same or different from the acetate kinase used as a nucleoside diphosphate kinase in the conversion of NDP to NQP synthesis or NTP synthesis. In some embodiments, the acetate kinase is a naturally occurring or wild-type acetate kinase. In some embodiments, the acetate kinase is an engineered acetate kinase, for example the engineered acetate kinase described herein.

Accordingly, in some embodiments, the NTP regenerating system includes pyruvate kinase and phosphoenolpyruvate. In some embodiments, the NTP regenerating system includes creatine kinase and creatine phosphate. In some embodiments, the NTP regenerating system includes polyphosphate kinase and inorganic polyphosphate. In some embodiments, the NTP regenerating system includes acetate kinase and acetyl phosphate.

In some embodiments, the NTP recycling system further includes additional enzymes (i.e., auxiliary enzymes) and substrates for regeneration of the phosphate donor used by the enzymatic conversion of NDP to NTP and/or do reduce byproducts of the NTP regenerating system. The enzyme for regeneration of the phosphate donor is selected based on the phosphate donor used in the conversion of NDP to NTP. By way example and not limitation, the enzyme for regeneration of the phosphate donor is pyruvate oxidase for regenerating acetyl phosphate. In some embodiments, the substrate for pyruvate oxidase is inorganic phosphate and pyruvate. In some embodiments, where the auxiliary enzyme is pyruvate oxidase, the NTP regeneration system further comprises catalase for removal of H₂O₂.

It is to be understood that for each and every method or process described for NQP or NTP synthesis, a NTP regenerating system can be incorporated into the enzymatic reactions or enzyme cascades.

### Enzymes for NQP/NTP Synthesis and Enzymatic Reactions

The enzymes for the process and methods described herein can use wild-type enzymes, engineered enzymes, and combinations of wild-type enzymes and engineered enzymes. Various combinations of such enzymes can be applied to the methods and processes as appropriate where such enzymes are used. Where appropriate for specific enzymes described below, the database accession numbers for the amino acid sequence, e.g., UniProt, NCBI, Genebank, etc., are provided in parentheses following the organism name.

Accordingly, in some embodiments, the present disclosure provides enzymes for the conversion of a nucleoside to a NMP via addition of a phosphate group to the 5′ position of the sugar. In some embodiments, the present disclosure provides enzymes for the conversion of a NMP to an NDP. In some embodiments, the present disclosure provides enzymes for the conversion of a NDP to an NTP.

In some embodiments, the present disclosure provide enzymes for the phosphorylation of the 3-OH of a nucleoside and a nucleotide. In some embodiments, the present disclosure provides enzymes for the conversion of a NTP to an NQP.

### Nucleoside Kinase/Adenosine Kinase

In some embodiments, nucleoside kinases are used for the conversion of a nucleoside to a nucleoside monophosphate (NMP). In the embodiments herein, any suitable nucleoside kinase may be used. Various suitable nucleoside kinases are known in the art. These include homologs of nucleoside kinases. In some embodiments, more than one nucleoside kinase can be used in the enzymatic reactions. In some embodiments, the nucleoside kinase useful for in the enzymatic reaction include, among others, the nucleoside kinase of Thermoplasma acidophilum (Q9HJT3), Methanocaldococcus jannaschii (Q57849), Burkholderia thailandensis (AIP24308.1), Thermus thermophilus (Q5SKR5), Caldilinea aerophila (WP_014432899.1), Geobacillus stearothermophilus (WP_043905564.1), Meiothermus ruber (WP_013014613.1), Thermostaphylospora chromogena (A0A1H1G3W4), Carbonactinospora thermoautotrophica (A0A132MV62), or Xanthomonas campestris (WP_011038567.1).

In some embodiments, the nucleoside kinase is an adenosine kinase. Various adenosine kinases can be used in the enzymatic reactions as the nucleoside kinase. In some embodiments, the adenosine kinase is a bacterial, fungal, plant, or animal adenosine kinase. In some embodiments, an adenosine kinase for use in the enzymatic reactions include, among others, the adenosine kinase of Chizosaccharomyces japonicus yFS275 (XP_002172336.1), Saccharomyces cerevisiae S288C (NP_012639.1), Drosophila melanogaster (NP_731676.2), Aradidopsis thaliana (NP_181262.1), Nicotiana tabacum (Q5DKU8), homo sapien (Q86U79), Thermostaphylospora chromogena (A0A1H1G3W4), Carbonactinospora thermoautotrophica (A0A132MV62), or Xanthomonas campestris (WP_011038567.1). In some embodiments, various adenosine kinase enzymes that may be used in the present invention are presented, below, in the Examples.

In some embodiments, the adenosine kinase is an engineered adenosine kinase. In some embodiments, the adenosine kinase is an adenosine kinase of an even numbered SEQ ID NO. of SEQ ID NOs: 66-1204, with or without amino acid residues 1-12 (i.e., His-tag), and is described in U.S. provisional application 63/589,818, filed Oct. 12, 2023, incorporated by reference herein. These engineered adenosine kinases exhibit improved properties compared to the parent wild-type enzyme, including among others, i) increased activity on unmodified nucleoside, ii) increased thermostability, iii) increased activity on cytidine, iv) increased activity on 2-fluoro modified 2′-deoxynucleosides, and/or v) increased activity on 2-O-methyl modified nucleosides.

### Nucleoside Monophosphate Kinase/Adenylate Kinase

In some embodiments, nucleoside monophosphate kinase (NMPK) is used for the conversion of nucleoside-5′-monophosphate (NMP) to nucleoside-5′-diphosphate (NDP). In the embodiments herein, any suitable nucleoside monophosphate kinase can be used. Various suitable nucleoside monophosphate kinases are known in the art. These include homologs of nucleoside monophosphate kinases. In some embodiments, more than one nucleoside monophosphate kinase can be used in the enzymatic reactions. In some embodiments, the nucleoside monophosphate kinase is an adenosine monophosphate kinase (e.g., adenylate kinase), cytidine monophosphate (CMP) kinase, uridine monophosphate (UMP) kinase, or guanylate-monophosphate (GMP) kinase.

In some embodiments, a nucleoside monophosphate kinase useful in the enzymatic reactions is a cytidine monophosphate kinases. Various suitable cytidine monophosphate kinases are known in the art. These include homologs of cytidine monophosphate kinases. In some embodiments, a cytidine monophosphate kinase useful in the enzymatic reactions includes, among others, the cytidine monophosphate kinase of Thermus thermophilus (Q5SL35), Pyrococcus furiosus (Q8U2L4), Pseudomonas putida (AF048857.1), Escherichia coli K-12 MG1655 (P0A6I0), Clostridium acetobutylicum (Q97I08), Halobacterium salinarum (Q9HPA5) Bacillus acidicola (WP_066270173), Acetobacter aceti (WP_010667744), Acidithiobacillus thiooxidans (WP_024892761.1), Acidithiobacillus ferrooxidans (WP_064220349.1), Metallosphaera sedula (WP_011921264.1), Amphibacillus xylanus (WP_015009966.1) Thioalkalivibrio denitrificans (WP_077278466.1), Vibrio psychroerythus (Q482G4), Pseudoalteromonas haloplanktis (Q3ILA1), Psychrobacter arcticus (Q4FRL5), Psychromonas ingrahamii (A1SZ01), Pseudomonas syringae (xQ4ZQ97) and Halobacterium salinarum (Q9HPA5).

In some embodiments, a nucleoside monophosphate kinase useful in the enzymatic reactions is a uridine monophosphate kinase. Various suitable uridine monophosphate kinases are known in the art. These include homologs of uridine monophosphate kinases. In some embodiments, a uridine monophosphate kinase useful in the enzymatic reactions includes, among others, the uridine monophosphate kinase of Pyrococcus furiosus (Q8U122), Thermus thermophilus (P43891), Pseudomonas putida (I7BW46), Escherichia coli K-12 MG1655 (P0A7E9), Aspergillus niger (A2R195), Saccharomyces cerevisiae (P15700), Clostridium acetobutylicum (Q97I64) ATCC 824 PyrH Halobacterium salinarum (Q9HNN8), Picrophilus torridus (WP_048059653), Metallosphaera sedula (WP_012021705), Thermoplasma acidophilum (WP_010900913), Sulfolobus solfataricus (WP_009992427), Acetobacter aceti (WP_042788648), Thioalkalivibrio sp. HK1 (WP_081759172.1), Amphibacillus xylanus (WP_015010200.1), Vibrio psychroerythus (Q485G8), Pseudoalteromonas haloplanktis (Q3IIX6), Psychrobacter arcticus (Q4FRH5), Psychromonas ingrahamii (ABM04676.1), Pseudomonas syringae (Q4ZWS6), and Halobacterium salinarum (Q9HNN8).

In some embodiments, the nucleoside monophosphate kinase useful in the enzymatic reactions is a guanosine monophosphate kinase (guanylate kinase). Various suitable guanylate kinases are known in the art. These include homologs of guanylate kinases. In some embodiments, a guanylate kinase useful in the enzymatic reactions includes, among others, the guanylate kinase of Thermotoga maritima (Q9X215), Thermus thermophilus (Q5SI18), Pseudomonas putida (I7C087), Escherichia coli K-12 (P60546), Aspergillus niger (A2QPV2), Saccharomyces cerevisiae (P15454), Clostridium acetobutylicum (Q97ID0), Acidithiobacillus ferrooxidans (WP_064219869.1), Acidithiobacillus thiooxidans (WP_010637919.1), Bacillus acidicola (WP_066264774.1), Acetobacter aceti (WP_018308252.1), Amphibacillus xylanus (WP_015010280.1), Thioalkalivibrio sulfidiphilus (WP_018953989.1), Vibrio psychroerythus (Q47UB3), Pseudoalteromonas haloplanktis (Q3IJH8), Psychrobacter arcticus (Q4FQY7), Psychromonas ingrahamii (A1T0P1), and Pseudomonas syringae (Q4ZZY8).

In some embodiments, the nucleoside monophosphate kinase useful in the enzymatic reactions is an adenosine monophosphate kinase (adenylate kinase). Various suitable adenylate kinases are known in the art. These include homologs of adenylate kinases. In some embodiments, the adenylate kinase is a bacterial, fungal, plant, or animal adenylate kinase. In some embodiments, an adenylate kinase useful in the enzymatic reactions includes, among others, adenylate kinases of Thermus thermophilus (Q72125), Pyrococcus furiosus (Q8U207), Pseudomonas putida (17CAA9), Escherichia coli K-12 W3110 (P69441), Aspergillus niger CBS 513.88 (A2QPN9), Saccharomyces cerevisiae (P07170), Clostridium acetobutylicum (Q97E39), Halobacterium salinarum (Q9HPAT), Acidithiobacillus thiooxidans (WP_024894015.1), Acidithiobacillus ferrooxidans (WP_064218420.1), Bacillus acidicola (WP_066267988.1), Sulfolobus solfataricus (WP_009991241.1), Saccharomyces cerevisiae (P07170), Thermotoga neapolitana (Q8GGL2), Escherichia coli (P69441) and Geobacillus stearothermophilus (WP_049624206.1). In some embodiments, various adenylate kinase enzymes that may be used in the present invention are presented, below, in the Examples.

In some embodiments, the adenylate kinase is an engineered adenylate kinase. In some embodiments, the adenylate kinase is an adenylate kinase of an even numbered SEQ ID NO. of SEQ ID NOs: 1206-2504, with or without amino acid residues 1-12 (i.e., His-tag), and is described in U.S. provisional application 63/589,828, filed Oct. 12, 2023, incorporated by reference herein. These engineered adenylate kinases exhibit improved properties compared to the parent wild-type enzyme, including among others, i) increased activity on unmodified nucleoside monophosphate (NMP), ii) increased stability, iii) increased activity on 2′-fluoro modified 2′-deoxynucleoside monophosphate, and iv) increased activity on 2′-O-methyl modified nucleoside monophosphate, or any combinations of i), ii), iii), and iv).

### Nucleoside Diphosphate Kinase/Acetate Kinase

In some embodiments, a nucleoside diphosphate kinase (NDPK) is used for the conversion of a nucleoside-5′-diphosphate (NDP) to the nucleoside-5-triphosphate. Various suitable nucleoside diphosphate kinases are known in the art. These include homologs of nucleoside diphosphate kinases. In some embodiments, a nucleoside diphosphate kinase useful in the enzymatic reactions includes, among others, the nucleoside diphosphate kinase of Aquifex aeolicus (067528), Pseudomonas putida (I7C0T7), Escherichia coli K-12 (P0A763), Aspergillus niger (A2QUJ6), Saccharomyces cerevisiae (P36010), Clostridium acetobutylicum (A6M162), Halobacterium salinarum (P61136), Acidithiobacillus thiooxidans (WP_024892623.), Acetobacter aceti (WP_042787791.1) Ndk Picrophilus (WP_011178084.1), Thermoplasma acidophilum (WP_010901523.1), Sulfolobus solfataricus (WP_009990482.1), Bacillus acidicola (WP_066262668.1), Ferroplasma (WP_009887649.1), Metallosphaera sedula (WP_011921175.1), Psychromonas ingrahamii (AlSZU8), Colwellia psychrerythraea (Q47WB6), Psychrobacter arcticus (Q4FTX1), Pseudoalteromonas haloplanktis (Q3ID15), Halobacterium salinarum (P61136), Natrialba magadii (D3SY02), Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), and Thermosipho africanus (WP_004102380.1).

In some embodiments, the nucleoside diphosphate kinase is an acetate kinase. Various suitable acetate kinase are known in the art. These include homologs of acetate kinase. In some embodiments, more than one acetate kinase is used. In some embodiments, one acetate is used for conversion of a NDP to the NTP product, and a different acetate kinase is used as a recycling enzyme for the conversion of NDPs to NTPs used as cofactors in the nucleoside kinase and nucleoside monophosphate kinase reactions, as described below.

In some embodiments, the acetate kinase for use in the enzymatic reactions is an acetate kinase of Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), and Thermosipho africanus (WP_004102380.1). In some embodiments, various acetate kinase enzymes that may be used in the present invention are presented, below, in the Examples.

In some embodiments, the acetate kinase is an engineered acetate kinase. In some embodiments, the acetate kinase is an acetate kinase of an even numbered SEQ ID NO. of SEQ ID NOs: 2506-3058, with or without amino acid residues 1-12 (i.e., His-tag), and is described in U.S. provisional application 63/589,839, filed Oct. 12, 2023, incorporated by reference herein. These engineered acetate kinases exhibit improved properties compared to the parent wild-type enzyme, including among others, i) increased activity on unmodified nucleoside diphosphate, ii) increased activity on 2′-fluoro modified 2′-deoxynucleoside diphosphate, and/or iii) increased activity on 2′-O-methyl modified nucleoside diphosphate.

### Polyphosphate Kinase PPK2

In some embodiments, a polyphosphate kinase can be used in place of the enzymatic actions of nucleoside monophosphate kinase and nucleoside diphosphate kinase, or a combination thereof. In a preferred embodiment, the polyphosphate kinase is PPK2. Various suitable PPK2s are known in the art. These include homologs of PPK2. In some embodiments, the PPK2 is the PPK2 of Deinococcus geothermalis DSM 11300 (WP_011531362.1), Meiothermus ruber DSM 1279 (ADD29239.1), Meiothermus silvanus DSM 9946 (WP_013159015.1), Thermosynechococcus elongatus BP-1 (NP_682498.1), Anaerolinea thermophila UNI-1 (WP_013558940), Caldilinea aerophila DSM 14535 (WP_014433181), Chlorobaculum tepidum ((NP_661973.1), Oceanithermus profundus DSM 14977 (WP_013458618), Roseiflexus castenholzii DSM 13941 (WP_012120763), Roseiflexus sp. RS-1 (WP_011956376), Truepera radiovictrix DSM 17093 (WP_013178933), Acidithiobacillus thiooxidans (WP_051690689.1), Acidithiobacillus ferrooxidans (WP_064219816.1), Psychrobacter arcticus (WP_083756052.1), Psychroserpens jangbogonensis (WP_033960485.1), Cryobacterium psychrotolerans (WP 092324020.1), Nocardioides psychrotolerans (WP_091116082.1), or Pseudomonas psychrophila (WP_019411115.1).

### 3′-O-Kinase

In some embodiments, a 3′-O-kinase (3OK) is used for the phosphorylation of a nucleoside or nucleotide (e.g., NMP, NDP, and NTP) at the 3′-OH of the sugar moiety. In some embodiments, any suitable 3′-O-kinase may be used. Various suitable 3′-O-kinase are known in the art. These include homologs of 3′-O-kinases, including homologs of CysC enzymes (adenylyl-sulfate kinase) and CoaE (dephospho-CoA kinase). CysC enzymes catalyze the conversion of adenosine 5′-phosphosulfate to 3′-phosphoadenylyl sulfate using ATP as a co-factor (C. Satishchandran et al., J. Biol. Chem., 1989, 264(25), 15012-15021). Similarly, CoaE enzymes are known to catalyze the conversion of 3′-dephospho-CoA to CoA, using ATP as a co-factor (Satishchandran C. et al. Biochemistry 1992, 31, 47, 11684-11688). In some embodiments, a 3′-O-kinase useful in the enzymatic reactions include, among others, the 3′O-kinase of E coli W3110 (P0A6J1), E coli W3110 (P0A6I9), Thermomonas hydrothermalis (A0A1M4SCV0), Geobacillus stearothermophilus (WP_033008536.1), Aquifex aeolicus (WP_010881297.1), Thermotoga sp. RQ7 (WP_041843859.1), Caldibacillus thermoamylovorans (A0A0D0ENB9), and Thermosynechococcus vestitus (Q8DJ87). In some embodiments, various acetate kinase enzymes that may be used in the present invention are presented, below, in the Examples.

In some embodiments, the 3′-O-kinase for the enzymatic reactions is an engineered 3′-O-kinase. In some embodiments, the 3′-O-kinase is a 3′-O-kinase of an even numbered SEQ ID NO. of SEQ ID NOs: 3060-3370 and 3376-5126, with or without amino acid residues 202-211 (i.e., His-tag), and is described in U.S. provisional application 63/387,908, filed Dec. 16, 2023, and International application No. PCT/US2023/076694, filed Oct. 12, 2023, incorporated by reference herein. These engineered 3′-O-kinases exhibit improved properties compared to the parent wild-type enzyme, including among others, including increased activity, increased activity on non-natural substrates, increased selectivity, increased substrate promiscuity, decreased product inhibition, and/or decreased byproduct formation.

### NTP Regeneration Enzymes

In some embodiments, the present disclosure also provides enzymes for NTP regeneration systems. Enzymes for NTP regeneration systems include, among others, pyruvate kinase, creatine kinase, polyphosphate kinase, and acetate kinase.

In some embodiments, the enzyme in the NTP regeneration system is a pyruvate kinase. Various suitable pyruvate kinases are known in the art. These include homologs of pyruvate kinase. In some embodiments, the pyruvate kinase useful in the NTP regenerating system includes, among others, pyruvate kinase of Escherichia coli str. K-12 substr. MG1655 (A0A3L1NNV5), Geobacillus stearothermophilus ATCC 7953 (Q02499), Schizosaccharomyces pombe (Q10208), Saccharomyces cerevisiae S288C (D6VPH8), Gallus gallus liver (F1NW43), Solanum tuberosum (P22200), Oryctolagus cuniculus M1/2 (O18919), Mus musculus muscle (P52480), Rattus norvegicus M1/2 (A0A8L2Q7B9), and Homo sapien muscle (A0A804F729).

In some embodiments, the enzyme in the NTP regeneration system is creatine kinase. Various suitable creatine kinases are known in the art. These include homologs of creatine kinase. In some embodiments, the creatine kinase useful in the NTP regenerating system includes, among others, the creatine kinase of Danio rerio (A8E5L0), Gallus gallus mitochondrial (P11009), Mus musculus (P07310), Bos taurus (Q5E9Y4), Oryctolagus cuniculus (P00563), and Homo sapien M-type (P06732).

In some embodiments, the enzyme in the NTP regeneration system is a polyphosphate kinase. Various suitable polyphosphate kinases are known in the art. These include homologs of polyphosphate kinases. In a preferred embodiment, the polyphosphate kinase is PPK1. In some embodiments, a PPK1 useful in the NTP regenerating system includes, among others, the PPK1 of Pseudomonas putida DOT-T1E (AFO50238.1), Escherichia coli K-12 P0A7B1 (AAC75554.1), Clostridium acetobutylicum ATCC 824 (NP_347259.1), Thermosynechococcus elongatus (WP_011056068), Acidithiobacillus ferrooxidans (WP_064219446), Acidithiobacillus thiooxidans (WP_031572361), Bacillus acidicola (WP_066264350), Acetobacter aceti (GAN58028), Acetobacter aceti (WP_077811826.1), Thioalkalivibrio denitrificans (WP_077277945.1), and Psychromonas ingrahamii (WP_041766473.1).

In some embodiments, the enzyme in the NTP regeneration system is an R-acetate kinase. As used herein, “R-acetate kinase” refers to the acetate kinase used in the NTP regenerating system. In some embodiments, the acetate kinase in the NTP regeneration system is the same acetate kinase used in the enzymatic conversion of a NDP substrate to the NTP product in the enzymatic cascades described herein. In some embodiments, the acetate kinase used in the NTP regeneration system is different from the acetate kinase used in the enzymatic conversion of a NDP substrate to the NTP product in the enzymatic cascades. Various suitable acetates kinases are known in the art and also describe herein. These include homologs of acetate kinases. In some embodiments, the acetate kinase useful in the NTP regeneration system includes, among others, an acetate kinase of Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), or Thermosipho africanus (WP_004102380.1). In some embodiments, various acetate kinase enzymes that may be used in the present invention are presented, below, in the Examples.

In some embodiments, the acetate kinase for use in the NTP regeneration system is an engineered acetate kinase. In some embodiments, the acetate kinase is an acetate kinase of an even numbered SEQ ID NO. of SEQ ID NOs: 2506-3058, with or without amino acid residues 1-12 (i.e., His-tag), and is described in U.S. provisional application 63/589,839, filed Oct. 12, 2023, incorporated by reference herein.

### Auxiliary Enzymes

In some embodiments, the present disclosure further provides auxiliary enzymes for the NTP regeneration system based on acetate kinase. In some embodiments, the auxiliary enzyme is a pyruvate oxidase when acetate kinase is present in the enzymatic cascades. Various pyruvate oxidases are known in the art. In some embodiments, the pyruvate oxidases are homologs of pyruvate oxidases. In some embodiments, the pyruvate oxidase is a pyruvate oxidase of Bifidobacterium mongoliense (A0A087C4V4), Alkalibacterium subtropicum (A0A1I1KLE2), Pisciglobus halotolerans (A0A1I3CCM7), Jeotgalibaca sp PTS2502 (A0A1U7E9W7), Vagococcus fluvialis (A0A2C9XUJ6), Candidatus Gracilibacteria bacterium (A0A2M7FGE0), Bavariicoccus seileri (A0A3D4S346), Bifidobacterium aquikefiri (A0A261G4D1), Aerococcus urinae (F2I8Y6), and Aerococcus suis (A0A1W1YA59).

In some embodiments, the auxiliary enzyme is a catalase. In some embodiments, where appropriate (e.g., use of acetate kinase and pyruvate oxidase), any suitable catalase may be used in the methods herein. Various suitable catalases are known in the art. In some embodiments, the catalase can be the catalase of Archaeoglobus fulgidus, Bacillus stearothermophilus, E. coli, Mycobacterium intracellulare, Synechococcus sp PCC7942, Arabidopsis thaliana, Pisum sativum, or Saccharomyces cerevisiae (see, e.g., Zamocky, M., Progress in Biophysics and Molecular Biology, 1999, 72(1):19-66); see also WO1992017571). In some embodiments, the catalase is a catalase of Bos taurus (P00432), Aspergillus niger (A0A254TZH3), Helicobacter pylori (J0N6C6), Drosophila melanogaster (P17336) and Rattus norvegicus (P04762).

In some embodiments, any of the enzymes described herein can be provided as a fusion protein. In some embodiments, the enzyme is fused to variety of polypeptide sequences, such as, by way of example and not limitation, polypeptide tags that can be used for detection and/or purification. In some embodiments, the fusion polypeptide of the enzyme comprises a glycine-histidine or histidine-tag (His-tag). In some embodiments, the fusion polypeptide of the enzyme comprises an epitope tag, such as c-myc, FLAG, V5, or hemagglutinin (HA). In some embodiments, the fusion polypeptide of the enzyme comprises a GST, SUMO, Strep, MBP, or GFP tag. In some embodiments, the fusion is to the amino (N-) terminus of engineered enzyme polypeptide. In some embodiments, the fusion is to the carboxy (C-) terminus of the enzyme polypeptide.

### Immobilized Enzymes

In some embodiments, it is further contemplated that any of the methods or processes using the enzyme/polypeptide can be carried out using the enzyme/polypeptide bound or immobilized on a substrate, such as a solid support, a porous substrate, a membrane, or particles. The polypeptide can be entrapped in matrixes or membranes. In some embodiments, matrices include polymeric materials such as calcium-alginate, agar, k-carrageenin, polyacrylamide, and collagen. In some embodiments, the solid matrices, includes, among others, activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the enzyme/polypeptide is immobilized on the surface of a support material. In some embodiments, the polypeptide is adsorbed on the support material. In some embodiments, the polypeptide is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, and ion-exchange resins, such as Amberlite, Sephadex, and Dowex.

In some embodiments, solid supports useful for immobilizing the enzyme/polypeptide in the present disclosure, include beads or resins comprising polymethacrylate with epoxide functional groups, polymethacrylate with amino epoxide functional groups, styrene/DVB copolymer or polymethacrylate with octadecyl functional groups. Exemplary solid supports useful for immobilizing the enzyme/polypeptide include, but are not limited to, EnginZyme (including, EziG-1, EziG-1, and EziG-3), chitosan beads, Eupergit C, and SEPABEADs (Mitsubishi) (including EC-EP, EC-HFA/S, EXA252, EXE119 and EXE120).

Methods of enzyme immobilization are known in the art. The engineered polypeptides can be bound non-covalently or covalently. Various methods for conjugation and immobilization of enzymes to solid supports (e.g., resins, membranes, beads, glass, etc.) are known in the art (see e.g., Yi et al., Proc. Biochem., 2007, 42(5): 895-898; Martin et al., Appl. Microbiol. Biotechnol., 2007, 76(4): 843-85; Koszelewski et al., J. Mol. Cat. B: Enzymatic, 2010, 63:39-44; Truppo et al., Org. Proc. Res. Dev., published online: dx.doi.org/10.1021/op200157c; Hermanson, Bioconjugate Techniques, 2nd Ed., Academic Press, Cambridge, M A (2008); Mateo et al., Biotechnol. Prog., 2002, 18(3):629-34; and “Bioconjugation Protocols: Strategies and Methods,” In Methods in Molecular Biology, Niemeyer (ed.), Humana Press, New York, NY (2004); the disclosures of each which are incorporated by reference herein).

### Reaction Conditions

In the embodiments provided herein and illustrated in the Examples, various components ranges of suitable reaction conditions that can be used in the processes, include but are not limited to, substrate loading, co-substrate loading, pH, temperature, buffer, solvent system, cofactor, polypeptide loading, and reaction time. Further suitable reaction conditions for carrying out the process for biocatalytic conversion of substrate compounds to product compounds using the enzymes described herein can be determined in view of the guidance provided herein includes, but is not limited to, contacting the enzymes and one or more substrate compounds under experimental reaction conditions of concentration, pH, temperature, and solvent conditions, and detecting the product compound. The reaction conditions described herein are illustrative and examples only. The present disclosure contemplates any suitable reaction conditions that may find use in the methods described herein.

The substrate compound, e.g., nucleoside and/or nucleotide, in the reaction mixtures can be varied, taking into consideration, for example, the desired amount of product compound, the effect of each substrate concentration on enzyme activity, stability of enzyme under reaction conditions, and the percent conversion of each substrate to product. In some embodiments, the suitable reaction conditions comprise a substrate compound loading of at least about 0.1 uM to 1 uM, 1 uM to 2 uM, 2 uM to 3 uM, 3 uM to 5 uM, 5 uM to 10 uM, or 10 uM or greater. In some embodiments, the suitable reaction conditions comprise a substrate compound loading of at least about 0.5 to about 25 g/L, 1 to about 25 g/L, 5 to about 25 g/L, about 10 to about 25 g/L, or 20 to about 25 g/L. In some embodiments, the suitable reaction conditions comprise a substrate compound loading of at least about 0.5 g/L, at least about 1 g/L, at least about 5 g/L, at least about 10 g/L, at least about 15 g/L, at least about 20 g/L, or at least about 30 g/L, or even greater.

In some embodiments, in carrying out the synthesis processes described herein, the enzymes/polypeptides can be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the enzyme(s) or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

In some embodiments, the gene(s) encoding the enzymes/polypeptides can be transformed into host cell separately or together into the same host cell. For example, in some embodiments one set of host cells can be transformed with gene(s) encoding one polypeptide and another set can be transformed with gene(s) encoding another polypeptide. Both sets of transformed cells can be utilized together in the reaction mixture in the form of whole cells, or in the form of lysates or extracts derived therefrom. In some embodiments, a host cell can be transformed with gene(s) encoding multiple polypeptides. In some embodiments the polypeptides can be expressed in the form of secreted polypeptides and the culture medium containing the secreted polypeptides can be used for the synthesis reaction.

In some embodiments, the enzyme/polypeptide, particularly any engineered enzyme disclosed herein, provides for processes wherein higher percentage conversion can be achieved with lower concentrations of the enzyme/polypeptide. In some embodiments of the process, the suitable reaction conditions comprise an enzyme/polypeptide amount of about 1% (w/w), 2% (w/w), 5% (w/w), 10% (w/w), 20% (w/w), 30% (w/w), 40% (w/w), 50% (w/w), 75% (w/w), 100% (w/w) or more of substrate compound loading.

In some embodiments, the enzyme/polypeptide is present at a molar ratio of enzyme/polypeptide to substrate of about 50 to 1, 25 to 1, 10 to 1, 5 to 1, 1 to 1, 1 to 5, 1 to 10, 1 to 25 or 1 to 50. In some embodiments, the enzyme/polypeptide is present at a molar ratio of enzyme/polypeptide to substrate from a range of about 50 to 1 to a range of about 1 to 50.

In some embodiments, the enzyme/polypeptide is present at about 0.01 g/L to about 50 g/L; about 0.01 to about 0.1 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the enzyme/polypeptide is present at about 0.01 g/L, 0.05 g/L, 0.1 g/L, 0.2 g/L, 0.5 g/L, 1, 2 g/L, 5 g/L, 10 g/L, 15 g/L, 20 g/L, 25 g/L, 30 g/L, 35 g/L, 40 g/L, or 50 g/L.

In some embodiments, as described herein, the suitable reaction conditions comprise a phosphate donor. In some embodiments, the phosphate donor used is selected for the enzymes used in the method or process. In some embodiments, the phosphate donor is a NTP (e.g., ATP, GTP, CTP, TTP, or UTP). In some embodiments, the phosphate donor includes acetyl phosphate, where acetate kinase is used in the method or process. In some embodiments, the phosphate donor is present at concentrations of about 1 to 500 uM; about 50 to 400 uM; about 100 to 300 uM; or about 200 to 300 uM. In some embodiments, the phosphate donor is regenerated or created by an enzyme, so that a lower concentration of phosphate donor is used.

In some embodiments, the reactions conditions is at a pH suitable for the enzymatic process. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In some embodiments, the pH of the reaction mixture may change during the course of the reaction. The pH of the reaction mixture may be maintained at a desired pH or within a desired pH range. In some embodiments, this may be done by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, borate, potassium phosphate, 2-(N-morpholino)ethanesulfonic acid (MES), 3-(N-morpholino)propanesulfonic acid (MOPS), acetate, triethanolamine, and 2-amino-2-hydroxymethyl-propane-1,3-diol (Tris), and the like. In some embodiments, the reaction conditions comprise water as a suitable solvent with no buffer present.

In the embodiments of the processes herein, a suitable temperature is used for the reaction conditions, for example, taking into consideration the increase in reaction rate at higher temperatures, and the activity of the enzyme during the reaction time period. Accordingly, in some embodiments, the suitable reaction conditions comprise a temperature of about 10° C. to about 95° C., about 10° C. to about 75° C., about 15° C. to about 95° C., about 20° C. to about 95° C., about 20° C. to about 65° C., about 25° C. to about 70° C., or about 50° C. to about 70° C. In some embodiments, the suitable reaction conditions comprise a temperature of about 10° C., 15° C., 20° C., 25° C., 30° C., 35° C., 40° C., 45° C., 50° C., 55° C., 60° C., 65° C., 70° C., 75° C., 80° C., 85° C., 90° C. or 95° C. In some embodiments, the temperature during the enzymatic reaction can be maintained at a specific, constant temperature throughout the course of the reaction. In some embodiments, the temperature during the enzymatic reaction can be adjusted over a temperature profile during the course of the reaction.

In some embodiments, the processes described herein are carried out in a suitable solvent. Suitable solvents include water, aqueous buffer solutions, organic solvents, polymeric solvents, and/or co-solvent systems, which generally comprise aqueous solvents, organic solvents and/or polymeric solvents. In some embodiments, the aqueous solvent (water or aqueous co-solvent system) may be pH-buffered or unbuffered. In some embodiments, the processes using the enzymes/polypeptides can be carried out in an aqueous co-solvent system comprising an organic solvent (e.g., ethanol, isopropanol (IPA), dimethyl sulfoxide (DMSO), dimethylformamide (DMF) ethyl acetate, butyl acetate, 1-octanol, heptane, octane, methyl t butyl ether (MTBE), toluene, and the like), ionic or polar solvents (e.g., 1-ethyl 4 methylimidazolium tetrafluoroborate, 1-butyl-3-methylimidazolium tetrafluoroborate, 1-butyl 3 methylimidazolium hexafluorophosphate, glycerol, polyethylene glycol, and the like). In some embodiments, the co-solvent can be a polar solvent, such as a polyol, dimethylsulfoxide (DMSO), or lower alcohol. In some embodiments, the non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the enzymes under the reaction conditions. Appropriate co-solvent systems can be identified by measuring the enzymatic activity of the specified enzymes with a defined substrate of interest in the candidate solvent system, utilizing an enzyme activity assay, such as those described herein.

In some embodiments of the process, the suitable reaction conditions comprise an aqueous co-solvent, where the co-solvent comprises DMSO at about 1% to about 50% (v/v), about 1 to about 40% (v/v), about 2% to about 40% (v/v), about 5% to about 30% (v/v), about 10% to about 30% (v/v), or about 10% to about 20% (v/v). In some embodiments of the process, the suitable reaction conditions can comprise an aqueous co-solvent comprising ethanol at about 1% (v/v), about 5% (v/v), about 10% (v/v), about 15% (v/v), about 20% (v/v), about 25% (v/v), about 30% (v/v), about 35% (v/v), about 40% (v/v), about 45% (v/v), or about 50% (v/v).

In some embodiments, the reaction conditions comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), TRITON™ X-100 polyethylene glycol tert-octylphenyl ether, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/mL, particularly from 1 to 20 mg/mL.

In some embodiments, the reaction conditions include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include Y-30® (Dow Corning), poly-glycol copolymers, oxy/ethoxylated alcohols, and polydimethylsiloxanes. In some embodiments, the anti-foam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the anti-foam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

The quantities or amounts of reactants used in the enzymatic reaction will generally vary depending on the quantities of product desired, and concomitantly the amount of substrates employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor, co-substrate and substrate may be added first to the solvent.

In some embodiments, solid reactants (e.g., substrate, enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

In some embodiments, for improved mixing efficiency when an aqueous co-solvent system is used, the polypeptide(s), and co-substrate may be added and mixed into the aqueous phase first. The substrate may be added and mixed in, followed by the organic phase or the substrate may be dissolved in the organic phase and mixed in. Alternatively, the substrate may be premixed in the organic phase, prior to addition to the aqueous phase.

In some embodiments, the processes of the present disclosure are generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like. In some embodiments, after suitable conversion to product, the reactants are separated from the product and additional reactants are added.

Any of the processes disclosed herein using the enzymes/polypeptides for the preparation of products can be carried out under a range of suitable reaction conditions, including but not limited to ranges of substrates, temperature, pH, solvent system, substrate loading, polypeptide loading, cofactor loading, and reaction time. In one illustrative example, the suitable reaction conditions for the conversion of a NDP or NTP to a NQP comprise: (a) substrate loading of about 1-200 mM NTP; (b) about 0.01 g/L to 5 g/L enzyme/polypeptide (e.g., 3′-O′-kinase, adenosine kinase, adenylate kinase, acetate kinase, etc.); (c) 1-100 mM MgCl₂; (e) 5 to 100 mM Tris-HCl buffer; (f) 10-100 mM LiKAcPO₄; (g) pH at 5-9; and (h) temperature of about 15° C. to 70° C. In another illustrative example, the suitable reaction conditions for the conversion of a NDP or NTP to a NQP comprise: (a) substrate loading of about 50 mM NDP or NTP; (b) about 0.01 g/L to 5 g/L enzyme/polypeptide (e.g., 3′-O′-kinase, acetate kinase, etc.); (c) 10 mM MgCl₂; (e) 50 mM Tris-HCl buffer; (f) 10 mM LiKAcPO₄; (g) pH 7.5; and (h) temperature of about 25° C. In some embodiments, the enzyme loading is between 1-30% w/w. In some embodiments, additional reaction components or additional techniques carried out to supplement the reaction conditions. These can include taking measures to stabilize or prevent inactivation of the enzyme, reduce product inhibition, shift reaction equilibrium to formation of the desired product.

In further embodiments, any of the above described processes for the conversion of one or more substrate compounds to product compound can further comprise one or more steps selected from: extraction; isolation; purification; and crystallization of product compound. Methods, techniques, and protocols for extracting, isolating, purifying, and/or crystallizing the product from biocatalytic reaction mixtures produced by the above disclosed processes are known to the ordinary artisan and/or accessed through routine experimentation. Additionally, illustrative methods are provided in the Examples below.

### EXAMPLES

The following Examples, including experiments and results achieved, are provided for illustrative purposes only and are not to be construed as limiting the present invention. Indeed, there are various suitable sources for many of the reagents and equipment described below. It is not intended that the present invention be limited to any particular source for any reagent or equipment item.

In the experimental disclosure below, the following abbreviations apply: M (molar); mM (millimolar), uM and μM (micromolar); nM (nanomolar); mol (moles); gm and g (gram); mg (milligrams); ug and μg (micrograms); L and 1 (liter); ml and mL (milliliter); ul, μl, uL, and μL (microliters); cm (centimeters); mm (millimeters); um and μιη (micrometers); sec. (seconds); min(s) (minute(s)); h(s) and hr(s) (hour(s)); U (units); MW (molecular weight); rpm (rotations per minute); psi and PSI (pounds per square inch); ° C. (degrees Celius); RT and rt (room temperature); CV (coefficient of variability); CAM and cam (chloramphenicol); PMBS (polymyxin B sulfate); IPTG (isopropyl β-D-1-thiogalactopyranoside); LB (lysogeny broth); TB (terrific broth); SFP (shake flask powder); CDS (coding sequence); DNA (deoxyribonucleic acid); RNA (ribonucleic acid); nt (nucleotide; polynucleotide); aa (amino acid; polypeptide); E. coli W3110 (commonly used laboratory E. coli strain, available from the Coli Genetic Stock Center [CGSC], New Haven, CT); HTP (high throughput); HPLC (high pressure liquid chromatography); HPLC-UV (HPLC-Ultraviolet Visible Detector); FIOPC (fold improvements over positive control) or FIOP (fold improvement over parent); Sigma and Sigma-Aldrich (Sigma-Aldrich, St. Louis, MO; Difco (Difco Laboratories, BD Diagnostic Systems, Detroit, MI); Microfluidics (Microfluidics, Westwood, MA); Life Technologies (Life Technologies, a part of Fisher Scientific, Waltham, MA); Amresco (Amresco, LLC, Solon, OH); Carbosynth (Carbosynth, Ltd., Berkshire, UK); Varian (Varian Medical Systems, Palo Alto, CA); Agilent (Agilent Technologies, Inc., Santa Clara, CA); Infors (Infors USA Inc., Annapolis Junction, MD);.

### Example 1

### Expression Enzymes and Purification in High Throughput (HTP)

### High Throughput (HTP) Growth of Enzymes and Variants Thereof

Transformed E. coli cells were selected by plating onto LB agar plates containing 1% glucose and 30 μg/ml chloramphenicol. After overnight incubation at 37° C., colonies were placed into the wells of 96-well shallow flat bottom NUNC™ (Thermo-Scientific) plates filled with 180 μl/well LB medium supplemented with 1% glucose and 30 μg/ml chloramphenicol. The cultures were allowed to grow overnight for 18-20 hours in a shaker (200 rpm, 30° C., and 85% relative humidity; Kuhner). Overnight growth samples (20 μL) were transferred into Costar 96-well deep plates filled with 380 μL of Terrific Broth supplemented with 30 μg/ml chloramphenicol. The plates were incubated for 120 minutes in a shaker (250 rpm, 30° C., and 85% relative humidity; Kuhner) until the OD₆₀₀ reached between 0.4-0.8. The cells were then induced with 40 μL of 10 mM IPTG in sterile water and incubated overnight for 18-20 hours in a shaker (250 rpm, 30° C., and 85% relative humidity; Kuhner). The cells were pelleted (4,000 rpm for 20 min), the supernatants were discarded, and the cells were frozen at −80° C. prior to analysis.

For lysis, 200 μL lysis buffer containing 50 mM Tris-HCl buffer, pH 7.5, and 0.1 g/L lysozyme were added to the cell pellet in each well. The cells were shaken vigorously at room temperature for 10 minutes on a bench top shaker. A 100 μL aliquot of the re-suspended cells was transferred to a 96-well format 200 μL BioRad PCR plate, then briefly spun-down prior to 1-hour heat treatment at the temperature indicated, typically 48-60° C. Following heat-treatment, the cell debris was pelleted by centrifugation (4,000 rpm, 4° C., 20 min), and clear supernatants were then used in biocatalytic reactions to determine their activity levels.

### Example 2

### Shake Flask Expression and Purification Procedure

### Shake Flask Expression

Method 1: Shake Flask Expression using IPTG induction. Selected HTP cultures, grown as described in Example 1, were plated onto LB agar plates with 1% glucose and 30 μg/mL chloramphenicol and grown overnight at 37° C. A single colony from each culture was transferred to 5 mL of LB broth with 1% glucose and 30 μg/mL chloramphenicol. The cultures were grown for 20 h at 30° C., 250 rpm, and subcultured at a dilution of approximately 1:50 into 250 mL of Terrific Broth with 30 μg/mL of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 μm PES membrane to further clarify the lysate.

Method 2: Shake Flask Expression using auto-induction. Selected HTP cultures, grown as described in Example 1, were plated onto LB agar plates with 1% glucose and 30 μg/mL chloramphenicol and grown overnight at 37° C. A single colony from each culture was transferred to 160 mL of Terrific Broth containing 0.075% glucose, 0.03% lactose, and 30 μg/mL of chloramphenicol. The cultures were grown for 20 h at 30° C. and 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.), and the supernatant was then filtered through a 0.2 μm PES membrane to further clarify the lysate.

Method 3: Shake-Flask Expression using IPTG induction and collection of enzyme as shake flask powder. Selected HTP cultures, grown as described in Example 1, were plated onto LB agar plates with 1% glucose and 30 μg/mL chloramphenicol and grown overnight at 37° C. A single colony from each culture was transferred to 5 mL of LB broth with 1% glucose and 30 μg/mL chloramphenicol. The cultures were grown for 20 h at 30° C., 250 rpm, and subcultured at a dilution of approximately 1:50 into 250 mL of Terrific Broth with 30 μg/mL of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (11,000 rpm for 60 min at 4° C.). The supernatant was collected in petri dishes and frozen at −80° C. The water was then removed under reduced pressure with a lyophilizer. The resultant powder was then collected and stored at −20° C.

Purification of from Shake Flask Lysates

Lysates were supplemented with 1/10^{th} volume of SF elution buffer (50 mM Tris-HCl, 500 mM NaCl, 250 mM imidazole, 0.02% v/v Triton X-100 reagent). Lysates were then purified using an AKTA Pure purification system and a 5 mL HisTrap FF column (GE Healthcare) using the run parameters in Table 7.1. The SF wash buffer comprised 50 mM Tris-HCl, 300 mM NaCl, 20 mM imidazole, 0.02% v/v Triton X-100 reagent.

TABLE 2.1
Purification Parameters

| Parameter | Volume | |
|---|---|---|
| Column volume | 5 | mL |
| Flow rate | 8 | mL/min |
| Pressure limit | 0.3 | mPa |
| Sample volume | 35 | mL |
| Equilibration volume | 5 column volumes (CV) = 25 mL | |
| Wash Unbound volume | 20 CV = 100 mL | |
| Elution | Isocratic (step) | |
| Elution volume | 5 CV = 25 mL | |
| Fraction volume | 1.5 | mL |

| | |
|---|---|
| RE-equilibration volume | 5 CV = 25 mL |

Elution fractions containing protein were identified by UV absorption (A280) and pooled, then dialyzed overnight in dialysis buffer (20 mM Tris-HCl, pH 7.4, 100 mM KCl, 0.1 mM EDTA, and 50% glycerol) in a 3.5K Slide-A-Lyzer™ dialysis cassette (Thermo Fisher) for buffer exchange. Protein concentrations in the preparations were measured by absorption at 280 nm, and preparations were stored at −20° C.

### Example 3

### High Performance Liquid Chromatography (HPLC) Analysis of Nucleotides

### Sample Preparation for Reaction Analysis Using HPLC:

Enzyme reactions were quenched by the addition of 4 volume equivalents (5× dilution) or 34 volume equivalents (35× dilution) of 75% v/v MeOH/water. The plate was sealed, mixed well, and centrifuged at 4,000 rpm for 4 min at 4° C. The supernatant was collected and analyzed by HPLC using an Ultimate 3000 system.

TABLE 3.1
Method 1: Achiral HPLC Parameters (Acetonitrile Gradient)

| | |
|---|---|
| Instrument | Ultimate 3000 HPLC System with a PAL autosampler |
| Column | Agilent Zorbax RR StableBond Aq, 150 × 3.0 mm × |
| | 3.5 μm |
| Guard Column | Agilent Zorbax StabeBond Aq, 5.0 × 3.0 mm, 1.8 μm |
| Mobile Phases | A: 50 mM sodium phosphate, 2 mM |
| | tetrabutylammonium bisulfate, pH 7.0 |
| | B: Acetonitrile |
| LC Gradient | 0-3.0 min: 0-−20% B |
| | 3.0-3.5 min: 20% B |
| | 3.5-4.0 min: 20-0% B |
| | 4.0-6.5 min: 0% B |
| Flow rate | 1.5 mL/min |
| Run time | 6.5 min |
| Column | 30° C. |
| temperature | |
| Injection volume | 10 μL |
| UV Detector | 254 nm |

TABLE 3.2
Method 2: Achiral HPLC Parameters (Ion Pairing Gradient)

| | |
|---|---|
| Instrument | Ultimate 3000 HPLC System with a PAL autosampler |
| Column | Agilent Zorbax RR StableBond Aq, 150 × 3.0 mm × |
| | 3.5 μm |
| Guard Column | Agilent Zorbax StabeBond Aq, 5.0 × 3.0 mm, 1.8 μm |
| Mobile Phases | A: 50 mM sodium phosphate, 2 mM |
| | tetrabutylammonium bisulfate, pH 7.0 |
| | B: Acetonitrile |
| | C: Water |
| LC Gradient | 0-4.0 min: 9.0% B, 48.5-16.2% C |
| | 4.0-4.5 min: 9.0% B, 16.2-48.5% C |
| | 4.5-5.5 min: 9.0% B, 48.5% C |
| Flow rate | 1.2 mL/min |
| Run time | 5.5 min |
| Column | 30° C. |
| temperature | |
| Injection volume | 10 μL |
| UV Detector | 254 nm |

TABLE 3.3
Method 3: Achiral HPLC Parameters (Isocratic)

| | |
|---|---|
| Instrument | Ultimate 3000 HPLC System with a PAL autosampler |
| Column | Agilent Zorbax RR StableBond Aq, 150 × 3.0 mm × |
| | 3.5 μm |
| Guard Column | Agilent Zorbax StabeBond Aq, 5.0 × 3.0 mm, 1.8 μm |
| Mobile Phases | A: 50 mM sodium phosphate, 2 mM |
| | tetrabutylammonium bisulfate, pH 7.0 |
| | B: Acetonitrile |
| LC Gradient | 0-4.0 min: 7.0% B |
| Flow rate | 1.2 mL/min |
| Run time | 4.0 min |
| Column | 30° C. |
| temperature | |
| Injection volume | 10 μL |
| UV Detector | 254 nm |

### Sample Preparation and Reaction Analysis Using HPLC

The nucleoside substrates, along with their respective 5′-monophosphate (NMP), 5′-diphosphate (NDP), and 5′-triphosphate (NTP) products produced using reactions set up as described in Example 4 were analyzed using HPLC. Mobile phases consisted of 50 mM potassium phosphate (pH 7) with 2 mM tetrabutylammonium hydrogen sulfate (Solvent A), acetonitrile (solvent B), and water (Solvent C). Products were detected by UV absorption at 254 nm. In some instances, a Zorbax RR StableBond Aq, 3.0×150 mm, 3.5 μm (Agilent, #863954-314) column was used. In other instances, a Zorbax RR StableBond Aq, 3.0×100 mm, 3.5 μm (Agilent, #861954-314) column was used, while in other instances, a Zorbax RR StableBond Aq, 2.1×50 mm, 3.5 μm (Agilent, #871700-914) column was used.

### Example 4

### Adenosine Kinases and Expression of Adenosine Kinase Enzymes

Synthetic genes encoding N-terminal 6-histidine tagged versions of three wild-type (WT) adenosine kinase enzymes (AdoK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the adenosine kinase (AdoK) expression constructs were grown at shake-flask scale using IPTG induction, as described in Example 2 (Method 1). The cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and adenosine kinase enzyme concentrations were measured by absorption at 280 nm using a NanoDrop™ 1000 spectrophotometer. Soluble protein concentrations are summarized in Table 4.1 below.

TABLE 4.1
Soluble Enzyme Production of Adenosine Kinase Variants

| | | FIOP Soluble |
|---|---|---|
| SEQ | | Enzyme Production |
| (ID NO: | Source organism of adenosine | (Relative to |
| nt/aa) | kinase gene sequence | SEQ ID NO: 50) |
| 1/2 | Thermostaphylospora chromogena | ++ |
| 3/4 | Carbonactinospora thermoautotrophica | ++ |
| 5/6 | Xanthomonas campestris | + |

Levels of increased soluble enzyme production were determined relative to the reference polypeptide of SEQ ID NO: 50 and defined as follows: ““+”” 1.00 to 1.19, “+” >1.19

Engineered adenosine kinases. The wild-type (WT) adenosine kinase (AdoK) enzyme encoded by the genome of Xanthemonas campestris (UniProt ID: A0A3E1LD95) was used to generate engineered adenosine kinase variants, and are described in U.S. provisional application No. 63/589,818, incorporated by reference herein. Briefly, a synthetic gene (SEQ ID NO: 5) encoding an N-terminal 6-histidine tagged version of the WT AdoK (SEQ ID NO: 6) was designed with codon optimization for E. coli expression, synthesized, and subcloned into the E. coli expression vector pCK100900i (See e.g., U.S. Pat. No. 7,629,157 and US Pat. Appln. Publn. 2016/0244787) all of which are hereby incorporated by reference). This plasmid construct was transformed into an E. coli strain derived from W3110. Directed evolution techniques generally known by those skilled in the art, e.g., saturation mutagenesis and recombination of identified beneficial mutations, were used to generate libraries of gene variants from these plasmids. The variants were selected under various assay conditions and selected for, e.g., for increased enzymatic activity, increased activity on different nucleosides, and increased activity on modified nucleosides. These engineered adenosine kinase enzymes have amino acid sequences comprising an even-numbered SEQ ID NO. of SEQ ID NOs: 66-1204.

### Example 5

### Adenylate Kinase and Expression of Adenylate Kinase Enzymes

Synthetic genes encoding N-terminal 6-histidine tagged versions of wild-type (WT) and evolved adenylate kinase enzymes (AdK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the adenylate kinase expression construct were grown at shake-flask scale, as described in Example 2, (Method 1). Cells were then lysed, purified, and dialyzed into storage buffer (20 mM Tris-HCl, pH 7.4, 100 mM KCl, 0.1 mM EDTA, and 50% glycerol), as described in Example 7. After overnight dialysis, protein samples were removed, and adenylate kinase enzyme concentrations were measured by absorption at 280 nm using a NanoDrop™ 1000 spectrophotometer. Soluble protein concentrations are summarized in Table 2.1Table 5.1 below.

TABLE 5.1
Soluble Enzyme Production of Variants
Relative to SEQ ID NO: 16

| | | FIOP Soluble |
|---|---|---|
| SEQ | | Enzyme Production |
| ID NO: | Source organism of AdyK | (Relative to |
| (nt/aa) | gene sequence | SEQ ID NO: 16) |
| 7/8 | Saccharomyces cerevisiae | +++ |
| 9/10 | Saccharomyces cerevisiae | +++ |
| 11/12 | Thermotoga neapolitana | ++ |
| 13/14 | Escherichia coli | ++ |
| 15/16 | Geobacillus stearothermophilus | + |

Levels of increased soluble enzyme production were determined relative to the reference polypeptide of SEQ ID NO: 16 and defined as follows: “+” 1.00 to 1.10, “++” >1.10, “+++” >2.10

Engineered adenylate kinases. The wild-type (WT) adenylate kinase (AdyK) enzyme (SEQ ID NO: 12) encoded by the genome of Thermotoga neapolitana (UniProt ID: Q8GGL2) was used to generate engineered adenylate kinase variants, and are described in U.S. provisional application No. 63/589,828, incorporated by reference herein. Briefly, a synthetic gene (SEQ ID NO: 11) encoding an N-terminal 6-histidine tagged version of the WT AdoK was designed with codon optimization for E. coli expression, synthesized, and subcloned into the E. coli expression vector pCK100900i (see, e.g., U.S. Pat. No. 7,629,157 and US Pat. Appln. Publn. 2016/0244787, all of which are hereby incorporated by reference). This plasmid construct was transformed into an E. coli strain derived from W3110. Directed evolution techniques generally known by those skilled in the art, e.g., saturation mutagenesis and recombination of identified beneficial mutations, were used to generate libraries of gene variants from these plasmids. The adenylate kinase variants were assayed under different assay conditions and selected for, e.g., for increased enzymatic activity, increased activity on different nucleoside monophosphates, and increased activity on modified nucleosides. These engineered adenylate kinase enzymes have an amino acid sequence comprising an even-numbered SEQ ID NO. of SEQ ID NOs: 1206-2504.

### Example 6

### Guanylate Kinase and Expression of Guanylate Kinase Enzyme

Synthetic genes encoding N-terminal 6-histidine tagged versions of a guanylate kinase enzyme (GuK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the guanylate kinase expression construct were grown at shake-flask scale, as described in Example 2, (Method 1). Cells were then lysed, purified, and dialyzed into storage buffer (20 mM Tris-HCl, pH 7.4, 100 mM KCl, 0.1 mM EDTA, and 50% glycerol), as described in Example 2. After overnight dialysis, protein samples were removed, and guanylate kinase enzyme concentrations were measured by absorption at 280 nm using a NanoDrop™ 1000 spectrophotometer. Soluble protein concentrations are summarized in Table 6.1 below.

TABLE 6.1
Soluble Enzyme Production of Guanylate Kinase Variant

| SEQ ID NO: | Source organism of GuK | Soluble Enzyme Concentration |
|---|---|---|
| (nt/aa) | gene sequence | After Purification [mg/mL] |
| 17/18 | Branchiostoma floridae | + |

Levels of increased soluble enzyme production are defined as follows: “+” >12

### Example 7

### Acetate Kinase (AcK) Genes and Plasmid Construction

A previously engineered acetate kinase enzyme (ACK-101) featuring an N-terminal 6-histidine tag (See e.g., PCT/US22/23039, which is hereby incorporated by reference in its entirety) was produced in shake flask using IPTG induction according to Example 2, Method 1.

In addition, synthetic genes encoding an N-terminal or C-terminal 6-histidine tagged version of multiple wild-type (WT) acetate kinase (AcK) enzymes were cloned into the pCK110900 vector system (See e.g., U.S. Pat. No. 9,714,437, which is hereby incorporated by reference in its entirety) and subsequently expressed in an E. coli strain derived from W3110.

Cells transformed with the AcK expression constructs were grown at shake-flask scale using IPTG induction (Example 2, Method 1). Cells were then lysed, clarified, and the soluble fractions were diluted 20-fold into solution containing 1×SDS-PAGE Running Buffer and Reducing Agent (Invitrogen). Samples were run on a 4-12% Bis-Tris SDS-PAGE gel and stained using AcquaStain Protein Gel Stain (Bulldog Bio). An image of the gel was analyzed using GelAnalyzer v19.1 to quantify the intensity of acetate kinase bands by densitometry. Soluble acetate kinase expression levels are summarized in Table 7.1, showing fold improvement in soluble expression level relative to the acetate kinase from Thermotogaceae bacterium (SEQ ID NO: 5128).

TABLE 7.1
Soluble Enzyme Production of Variants
Relative to SEQ ID NO: 5128

| | | FIOP Soluble Enzyme |
|---|---|---|
| SEQ ID NO: | Source organism of | Expression |
| (nt/aa) | AcK gene sequence | (Relative to SEQ ID NO: 5128) |
| 19/20 | Marinitoga sp. 38H-ov | ++ |
| 21/22 | Thermotoga sp. KOL6 | + |
| 23/24 | Thermosipho melaniensis | + |
| 25/26 | Thermotoga sp. RQ7 | + |
| 27/28 | Thermosipho africanus | + |

Levels of increased soluble enzyme production were determined relative to the reference polypeptide of SEQ ID NO: 5128 and defined as follows: “+” 1.12 to 1.22, “+” >1.22.

Engineered acetate kinases. The wild-type (WT) acetate kinase (AcK) enzyme (SEQ ID NO: 20) encoded by the genome of Marinitoga sp. 38H-ov was used to generate engineered acetate kinase variants, and are described in U.S. provisional application No. 63/589,839, incorporated by reference herein. Libraries of genes were produced from the parent gene using various techniques, e.g. saturation mutagenesis and recombination of previously identified beneficial mutations. The acetate kinase variants were assayed under different assay conditions and selected for, e.g., for increased enzymatic activity, increased activity on different nucleoside diphosphates, and increased activity on modified nucleoside diphosphates. These engineered acetate kinase enzymes have an amino acid sequence of an even-numbered SEQ ID NO. of SEQ ID NOs: 2506-3058.

### Example 8

### Pyruvate Oxidase and Expression of Pyruvate Oxidase Enzyme

Synthetic genes encoding N-terminal 6-histidine tagged versions of wild-type (WT) pyruvate oxidase (POX) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the pyruvate oxidase expression construct were grown at shake-flask scale and the expressed enzymes were collected as lyophilized powders as described in Example 2, (Method 3). The relative expression levels of these enzymes were determined by gel electrophoresis. The relative expression levels as measured by gel electrophoresis are shown in Table 8.1 below.

TABLE 8.1
Enzyme Production of Pyruvate Oxidase Variants

| SEQ ID NO: | Source organism of pyruvate | Relative Enzyme |
|---|---|---|
| (nt/aa) | oxidase gene sequence | expression level |
| 29/30 | Bifidobacterium mongoliense | +++ |
| 31/32 | Alkalibacterium subtropicum | ++ |
| 33/34 | Pisciglobus halotolerans | ++ |
| 35/36 | Jeotgalibaca sp PTS2502 | ++ |
| 37/38 | Vagococcus fluvialis | ++ |
| 39/40 | Candidatus Gracilibacteria bacterium | ++ |
| 41/42 | Bavariicoccus seileri | ++ |
| 43/44 | Bifidobacterium aquikefiri | + |
| 45/46 | Aerococcus urinae | + |
| 47/48 | Aerococcus suis | + |

Levels of increased enzyme production were qualitatively determined and defined as follows: “+” low expression, “++” moderate expression, “+++” high expression

### Example 9

### 3′-O-Kinase (3OK) and Expression of 3′-O-Kinase Enzymes

Synthetic genes encoding an N-terminal or C-terminal 6-histidine tagged version of multiple wild-type (WT) 3′O-Kinase (3OK) enzymes were cloned into the pCK110900 vector system (See e.g., U.S. Pat. No. 9,714,437, which is hereby incorporated by reference in its entirety) and subsequently expressed in an E. coli strain derived from W3110.

Cells transformed with the 3OK expression constructs were grown at shake-flask scale using either IPTG induction (SEQ ID NOs: 49 and 51) or auto-induction (SEQ ID NOs: 53-63), as described in Example 2, (Methods 1 and 2 respectively). Cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and 3OK concentrations were measured by absorption at 280 nm using a NanoDrop™ 1000 spectrophotometer. Soluble protein concentrations are summarized in Table 9.1 below, showing a fold improvement in soluble protein production following shake-flask purification relative to the 3′O-kinase from Thermosynechococcus vestitus (SEQ ID NO: 64).

TABLE 9.1
Soluble Enzyme Production of Variants
Relative to SEQ ID NO: 64

| | | FIOP Soluble |
|---|---|---|
| SEQ | | Enzyme Production |
| ID NO: | Source organism of 3OK | (Relative to |
| (nt/aa) | gene sequence | SEQ ID NO: 64) |
| 49/50 | E coli W3110 | +++ |
| 51/52 | E coli W3110 | +++ |
| 53/54 | Thermomonas hydrothermalis | +++ |
| 55/56 | Geobacillus stearothermophilus | ++ |
| 57/58 | Aquifex aeolicus | ++ |
| 59/60 | Thermotoga sp. RQ7 | ++ |
| 61/62 | Caldibacillus thermoamylovorans | + |
| 63/64 | Thermosynechococcus vestitus | + |

Levels of increased soluble enzyme production were determined relative to the reference polypeptide of SEQ ID NO: 64 and defined as follows: “+” 1.00 to 6.40, “++” >6.40, “+++” >21.60

### Screening of WT 3OK Homologs for Activity on ATP

The eight 3OK WT homologs were produced in shake flask and purified, as described in Example 2. ACK-101, a previously engineered acetate kinase enzyme featuring an N-terminal 6-histidine tag (See e.g., PCT/US22/23039, which is hereby incorporated by reference in its entirety) was produced in shake flask using IPTG induction according to Example 2, Method 1.

The 3OK homologs were screened for conversion of ATP to AQP. Reactions were performed at 100 μL scale in Costar 96-well deep plates. Reactions included 1 mM ATP, 10 mM LiKAcPO₄, 1 mM MgCl₂, 0.2 g/L ACK-101, 0.5 g/L 3OK, in 50 mM Tris-HCl (pH 7.5). The reactions were set up by sequential addition of 5× stocks prepared in 50 mM Tris-HCl (pH 7.5) as follows: (i) 20 μL of a 1.0 g/L ACK-101 stock was added; (ii) 20 μL of a 50 mM LiKAcPO₄ stock was added; (iii) 20 μL of a 2.5 g/L stock of 3OK purified enzyme variant was added; (iv) 20 μL of a 5 mM MgCl₂ was added; (v) 20 μL of a 5 mM ATP stock was added. After mixing well and briefly centrifuging to collect the contents in the bottom of each well, the reaction plates were heat-sealed with a peelable aluminum seal and incubated for 18 h (400 rpm, 30° C.).

Reactions were analyzed by HPLC Method 1, as described in Example 3, and the results for reaction with ATP to produce AQP are shown in their respective columns in Table 9.2.

TABLE 9.2
Conversion of 3OKs with Native ATP

| SEQ ID NO: | Source organism of 3OK | Conversion of |
|---|---|---|
| (nt/aa) | gene sequence | ATP to AQP |
| 49/50 | E coli W3110 | ++ |
| 51/52 | E coli W3110 | ++ |
| 53/54 | Thermomonas hydrothermalis | |
| 55/56 | Geobacillus stearothermophilus | ++ |
| 57/58 | Aquifex aeolicus | + |
| 59/60 | Thermotoga sp. RQ7 | +++ |
| 61/62 | Caldibacillus thermoamylovorans | +++ |

Levels of conversion of ATP to 3′-phosphorylated ATP (AQP) are defined as follows: “+” 0.6-4.5 percent conversion, “++” >4.5 percent conversion, “+++” >41 percent conversion

Engineered 3OK variants. The wild-type 3′-O-kinase of Geobacillus stearothermophilus with a His-tag (SEQ ID NO: 56) was selected as the parent 3′-O-kinase enzyme for generating engineered 3′-O-kinase variants, as described in U.S. provisional application No. 63/387,908, filed Dec. 16, 2022, and PCT application No. PCT/US2023/076694, filed Oct. 12, 2023, all of which are incorporated by reference herein. Libraries of genes were produced from the parent gene (SEQ ID NO: 55) using various techniques, e.g. saturation mutagenesis and recombination of previously identified beneficial mutations, and the 3′-O-kinase variants assayed in presence of acetate kinase AcK 101 and NDP under different assay conditions and selected for, e.g., for increased enzymatic activity, increased activity in presence of different nucleoside diphosphates, and increased activity on modified nucleoside diphosphates. These engineered 3′-O-kinase enzymes have an amino acid sequence of an even-numbered SEQ ID NO. of SEQ ID NOs: 3060-3370 and 3376-5126.

### Example 10

POX-Driven Enzymatic Synthesis of ATP from Adenosine without Addition of Acetyl Phosphate

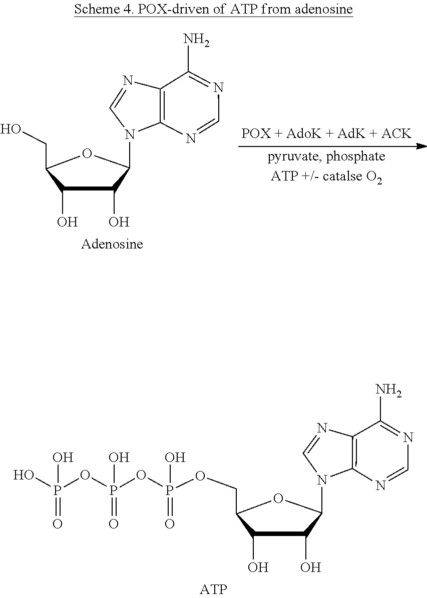

ACK-101 was produced and purified, as described in Example 2 (Method 3). AdoK (SEQ ID NO: 5/6) was produced and purified, as described in Example 3. AdK (SEQ ID NO: 11/12) was produced and purified, as described in Example 2 (Method 3). Pyruvate Oxidase (POX) (SEQ ID NO: 37/38) was produced and purified, as described in Example 2 (Method 3) and tested in a kinase cascade. Use of POX precludes the need for added LiKAcPO₄ in the kinase cascade.

Reactions were performed in 1.1 mL Axygen deepwell plates. As depicted in Scheme 4, reactions included 0.91 mM adenosine, 0.009 mM ATP, 9.1 mM MgSO₄, 1 g/L ACK-101, 0.4 g/L AdK (SEQ ID NO: 12), 0.46 g/L AdoK (SEQ ID NO: 6), 9.1 mM Tris-HCl (pH 8), 0.45 mM flavin adenine dinucleotide (FAD), 0.45 mM thiamine pyrophosphate (ThPP), 45.5 mM sodium pyruvate, 18 mM K₂HPO₄, and 0.26 g/L POX (SEQ ID NO: 38).

All reagents were dissolved in water. The reactions were prepared as follows: to a well was added sequentially by micropipette 10 μL 100 mM MgSO₄, 10 μL 100 mM Tris-HCl (pH 8), 10 μL 200 mM K₂HPO₄, 10 μL 10 mM adenosine 5 μL 10 mM FAD, 5 μL 10 mM ThPP, 10 μL 0.1 mM ATP, 10 μL 11.5 mg/mL ACK-101, 10 μL 4.8 mg/mL AdK (SEQ ID NO: 11/12), 10 μL 5.04 mg/mL AdoK (SEQ ID NO: 5/6), 10 μL 0.26 mg/mL POX (SEQ ID NO: 37/38), and 10 μL 500 mM pyruvate. The plate was sealed with a porous aeroseal and shaken at 300 rpm in an incubator set at 30° C. and 85% humidity for 17 hours.

Subsequently, the reactions were quenched by transferring 60 μL of the reaction mixture into 60 μL methanol. The quenched mixture was filtered with a 0.45 μm low-binding hydrophilic PTFE plate. A 20-μL aliquot of the filtrate was then diluted with 180 μL water. These samples were then analyzed by HPLC Method 2—ion pairing gradient in Example 3, and the results are shown in Table 10.1.

TABLE 10.1
POX-driven synthesis of ATP from adenosine

| POX SEQ ID NO: (nt/aa) | Percent conversion to ATP |
|---|---|
| 37/38 | + |

Levels of conversion of adenosine to ATP are defined as follows: “+” >75 percent conversion

### Example 11

Enzymatic Synthesis of NQP from Nucleoside

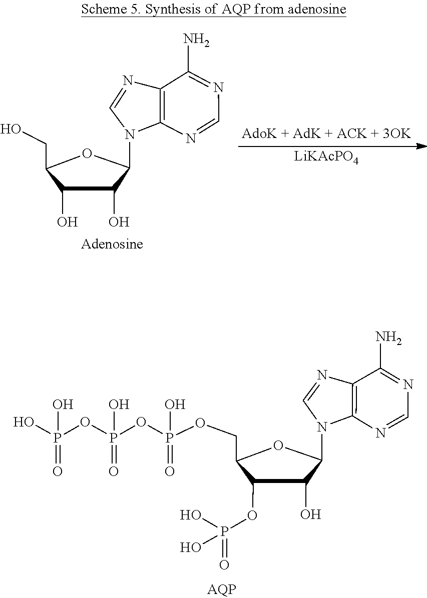

AcK-101 was produced and purified, as described in Example 2 (Method 3). AdoK (SEQ ID NO: 5/6) was produced and purified, as described in Example 2 (Method 1). AdK (SEQ ID NO: 11/12) was produced and purified, as described in Example 2 (Method 1). 3OK enzyme (SEQ ID NO: 59/60) were produced as described in Example 2 (Method 1) and tested in a kinase cascade resulting in the conversion of substrate nucleoside to the respective NQP.

Reactions were performed in 200 μL BioRad PCR plates. Reactions included 1.11 mM nucleoside, 11.1 mM LiKAcPO₄, 11.1 mM MgSO₄, 1.3 g/L ACK-101, 1.1 mg/mL 3OK (SEQ ID NO: 55/56 or SEQ ID NO: 59/60), 0.5 g/L AdK (SEQ ID NO: 11/12), 2.8 g/L AdoK (SEQ ID NO: 5/6), and 11.1 mM Tris-HCl (pH 8).

All reagents were dissolved in water. The reactions were prepared as follows: to a well was added sequentially by micropipette 10 μL 100 mM MgSO₄, 10 μL 100 mM Tris-HCl (pH 8), 10 μL 0.1 mM ATP, 10 μL 10 mM nucleoside 10 μL 11.5 mg/mL ACK-101, 10 μL 4.8 mg/mL AdK (SEQ ID NO: 12), 10 μL 25 mg/mL AdoK (SEQ ID NO: 6), 10 μL 10 mg/mL 3OK enzyme (SEQ ID NO: 56 or SEQ ID NO: 60), and then 10 μL 100 mM LiKAcPO₄. The plate was sealed and shaken at 400 rpm in an incubator set at 30° C. for 24 hours.

Subsequently, the reactions were quenched by removing 30 μL of the reaction mixtures, and four volume equivalents of 75% MeOH/Water (120 μL, 5× dilution) were added, as described in Example 3. Samples were analyzed by HPLC Method 2—ion pairing gradient and the results are shown in Table 11.1.

TABLE 11.1
Reaction of 3OK enzyme variants with ATP

| SEQ ID NO: | | |
|---|---|---|
| (nt/aa) | NTP Substrate | Percent Conversion to NQP |
| 59/60 | ATP | + |

Level of conversion of ATP substrate to AQP are defined as follows: “+” >1.5 percent conversion

### Example 12

### Relative Activities of AdoK for the Conversion of Nucleosides to Nucleotides in Enzyme Cascade

### Shake Flask Characterization of AdoK Variants

AdoK variants (SEQ ID NOs: 6, 152, 316, 432, 796, and 926) were expressed and purified as described in Example 2.

To assess activity, each variant was added to a 5 μL reaction at a final concentration of 10 μM. The reaction contained 50 mM Tris (pH 8.0), 50 mM lithium potassium acetyl phosphate, 10 μM ATP, 10 mM MgCl₂, 10 μM SEQ ID NO: 2234 (AdyK), 10 μM SEQ ID NO: 2880 (AcK), and 10 mM nucleoside. Reactions were incubated in a Multitron (Infors) shaker at 30° C. & 400 rpm for 60 min. Reactions were then quenched and diluted 40-fold with 75% methanol and analyzed by HPLC as described in Example 3. Relative activities were normalized to the lowest observed activity by a variant on a given substrate. The results are shown in Table 12.1.

TABLE 12.1

| | Relative Activities of AdoK Variants | | | | | | | | |
|---|---|---|---|---|---|---|---|---|---|
| SEQ ID NO: | on Nucleoside Substrates | | | | | | | | |
| (nt/aa) | A | C | G | U | fA | fC | fG | fU | mA |
| 5/6 | ~ | ~ | ++ | + | + | − | ++ | − | − |
| 151/152 | ~ | ~ | ++ | + | ++ | − | +++ | − | − |
| 315/316 | ~ | ~ | ++ | ~ | ~ | − | ~ | − | − |
| 431/432 | ~ | ~ | ++ | + | ++ | ~ | ++ | ~ | ~ |
| 795/796 | ~ | ~ | ++ | + | ++ | ++ | +++ | ++ | +++ |
| 925/926 | ~ | ~ | ~ | + | ++ | +++ | +++ | +++ | +++ |

Levels of relative activity were measured for the listed variants and defined as follows:
“−” 0.00 to 0.99,
“~” ≥ 1.0,
“+” > 1.06,
“++” > 2.00,
“+++” > 29.90.

### Example 13

### Relative Activities of AdyK for the Conversion of Nucleosides to Nucleotides in Enzyme Cascade

### Shake Flask Characterization of AdyK Variants

AdyK variants (SEQ ID NOs: 12, 1374, 1710, 1786, 1876, 2076, and 2234) were expressed and purified as described in Example 2.

To assess activity, each variant was added to a 5 μL reaction at a final concentration of 10 μM. The reaction contained 50 mM Tris (pH 8.0), 50 mM lithium potassium acetyl phosphate, 10 μM ATP, 10 mM MgCl₂, 10 μM SEQ ID NO: 958, 10 μM SEQ ID NO: 2888, and 10 mM nucleoside. Reactions were incubated in a Multitron (Infors) shaker at 30° C. & 400 rpm for 60 min. Reactions were then quenched and diluted 40-fold with 75% methanol and analyzed by HPLC as described in Example 3. Relative activities were normalized to the lowest observed activity by a variant on a given substrate. The results are shown in Table 13.1.

TABLE 13.1

| | Relative Activities of AdyK Variants on | | | | | | | | |
|---|---|---|---|---|---|---|---|---|---|
| SEQ ID NO: | Nucleoside Substrates | | | | | | | | |
| (nt/aa) | A | C | G | U | fA | fC | fG | fU | mA |
| 11/12 | ~ | ~ | ~ | ~ | ~ | − | ~ | − | + |
| 1373/1374 | ~ | ++ | ++ | +++ | ~ | ~ | + | − | + |
| 1709/1710 | ~ | ++ | ++ | +++ | ~ | + | ++ | + | + |
| 1785/1786 | ~ | ++ | ++ | +++ | ~ | + | ++ | ~ | + |
| 1875/1876 | ~ | ++ | ++ | +++ | ~ | + | ++ | + | + |
| 2075/2076 | ~ | ++ | ++ | +++ | ~ | + | +++ | + | + |
| 2233/2234 | ~ | ++ | ++ | +++ | ~ | + | +++ | + | ~ |

Levels of relative activity were measured for the listed variants and defined as follows:
“−” 0.00 to 0.99,
“~” ≥ 1.0,
“+” > 1.10,
“++” > 4.15,
“+++” > 100.00.

### Example 14

### Relative Activities of AcK for the Conversion of Nucleosides to Nucleotides in Enzyme Cascade

### Shake Flask Characterization of AcK Variants

AcK variants_SEQ ID NOs 20, 2746, and 2888 were expressed and purified as described in Example 2.

To assess activity, each variant was added to a 5 μL reaction at a final concentration of 10 μM. The reaction contained 50 mM Tris (pH 8.0), 50 mM LiK(acetyl phosphate), 10 μM ATP, 10 mM MgCl₂, 10 μM SEQ ID NO: 958, 10 μM SEQ ID NO: 2234, and 10 mM nucleoside. Reactions were incubated in a Multitron (Infors) shaker at 30° C. & 400 rpm for 60 minutes. Reactions were then quenched and diluted 40-fold with 75% methanol and analyzed by HPLC as described in Example 3. Relative activities were normalized to the lowest observed activity by a variant on a given substrate. The results are shown in Table 14.1.

TABLE 14.1

| | Relative Activities of AK Variants on | | | | | | | | |
|---|---|---|---|---|---|---|---|---|---|
| SEQ ID NO: | Nucleoside Substrates | | | | | | | | |
| (nt/aa) | A | C | G | U | fA | fC | fG | fU | mA |
| 19/20 | ~ | ~ | ~ | ~ | ~ | ~ | ~ | ~ | ~ |
| 2745/2746 | ~ | ~ | + | ~ | ~ | ++ | + | ++ | +++ |
| 2887/2888 | ~ | ~ | + | ~ | ~ | ++ | ++ | ++ | +++ |

Levels of relative activity were measured for the listed variants and defined as follows:
“~” ≥ 1.0,
“+” > 1.10,
“++” > 1.60,
“+++” > 2.00.

### Example 15

### Relative Activities of 3OK for the Conversion of Nucleosides to Nucleotides

3′-O-kinases of SEQ ID NOs: 56, 3376, 3500, 4468, 4804, 5082, and 5118 were selected as the parents of 3OK enzymes. Seven shake-flask parent variants were grown, expressed, and purified as described in Example 2, Method 2

Reactions were performed at 2 μL scale in 96-well format 200 μL BioRad PCR plates. Reactions included substrate, LiKAcPO₄, MgCl₂, AcK101, 25% 3OK solution, in Tris-HCl. The reactions were set up as follows: (i) all reaction components except for 3OK solution were pre-mixed in a single solution, and 1.5 μL of this solution was aliquoted into each well of the 96-well plate; (ii) 0.5 μL 3OK solution was added to each well. After briefly centrifuging to collect the contents in the bottom of each well, the reaction plates were heat-sealed with a peelable aluminum seal and incubated at 30° C. for indicated hours, then held at 4° C. until the reaction was quenched by 6 μL of MeOH. Quenched reaction mixtures were then subject to a TdT coupled assay for detection of NQP products. Evolved TdT (as described in U.S. provisional application No. 63/387,908, filed Oct. 13, 2022, and PCT application No. PCT/US2023/76694, filed Oct. 12, 2023, all of which are incorporated by reference herein) will couple a fluorescently labelled oligo with an NQP donor, producing a distinct product detectable by capillary electrophoresis. The reaction mixture was further diluted to 40 μM by water, then coupled with FAM labeled TdT oligo as described in Table 15.1. The reaction plates were heat-sealed with a peelable aluminum seal and incubated at 30° C. for 1 h and 95° C. for 2 min, then held at 4° C. until the reaction was quenched.

After quenching and sample preparation as described below, reactions were analyzed.

TABLE 15.1
Reaction, quench, and analytical properties

Reaction conditions: Substrate 10 mM ATP, 10 mM GTP, 10 mM CTP,
10 mM UTP, 1 mM fATP, 1 mM GTP-αS; Reaction buffer - 100 mM
Tris-HCl, pH 8, 50 mM LiKAcPO₄, 10 mM Magnesium chloride, 0.2
g/L AcK101; 3OK - 39 uM; Reaction time - 16 hour.
TdT coupling reaction conditions: Substrate - 10 μM SEQ ID: 5129,
10 μM above reaction mixture; Reaction buffer - 20 mM TEoA, pH 7.8,
50 mM CoCl₂, 0.001 U/μL IPP; Enzyme: 4 μM SEQ ID: 3374
(TdT variant)
Quench conditions: Reactions were quenched, and sample preparation as described below for capillary electrophoresis

Sample preparation for reaction analysis using CE: Reaction samples were analyzed via capillary electrophoresis using an ABI 3500xl Genetic Analyzer (ThermoFisher). Reactions (2 μL) were quenched by the addition of 38 μL of 10 mM aqueous EDTA. The quenched reaction mixture was further diluted 80000 times by water. 2 μL of this quenched solution was transferred to a new 96-well MicroAmp Optical PCR plate or 384-well MicroAmp Optical PCR plate containing 18 μL Hi-Di™ Formamide (ThermoFisher) which has an appropriate size standard. The ABI3500xl was configured with POP6 polymer, 50 cm capillaries and a 45° C. oven temperature. Pre-run settings were 18 KV for 180 sec. Injection was 5 KV for 5 sec, and the run settings were 19.5 KV for 640 sec. FAM-labeled oligo substrates and products (SEQ ID NOs: 5129-5135) were identified by their sizes relative to the sizing ladder, with the substrate oligo peak at ˜18 or 20 bp and the products appearing in the region of ˜14-18 bp.

Activities of 3OK variants SEQ IDs: 56, 3376, 3500, 4468, 4804, 5082, and 5118 with NTP substrates were measured as follows: Activity (% conversion) were calculated as 3′PO₄ product peak areas (SEQ ID NOs: 5130-5135) over the total of the unreacted substrate (SEQ ID: 5127, byproduct, and 3′PO₄ product peak areas). The results are shown in Table 15.2.

TABLE 15.2
conversion of nucleotide triphosphates to 3′ phosphorylated products

| | Relative Activities of 3OKinase | | | | | |
|---|---|---|---|---|---|---|
| SEQ ID No: | variants on nucleotide substrates | | | | | |
| (nt/aa) | CTP | UTP | GTP | ATP | GTP-αS | fATP |
| 55/56 | − | − | − | + | − | − |
| 3375/3376 | − | − | + | +++ | − | − |
| 3499/3500 | + | + | ++ | +++ | + | − |
| 4467/4468 | ++ | ++ | +++ | +++ | + | + |
| 4803/4804 | ++ | ++ | +++ | +++ | +++ | + |
| 5081/5082 | +++ | +++ | +++ | ++++ | +++ | + |
| 5117/5118 | +++ | +++ | +++ | ++++ | +++ | ++ |

Levels of relative activity were measured for the listed variants and defined as follows:
“−” 0.00 to 0.99,
“+” ≥ 1.0,
“++” > 10.0,
“+++” > 30.0.
“++++” > 70.0.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

### Appendix

APPENDIX TABLE A1
Description of modifications in oligonucleotide sequences

| Alias | Description |
|---|---|
| * | there is a phosphorothioate bond between these bases |
| A[n] | poly-2′-deoxyadenosine of length n |
| C[n] | poly-2′-deoxycytidine of length n |
| G[n] | poly-2′-deoxyguanosine of length n |
| T[n] | poly-2′-deoxythymidine of length n |
| 5′-6-FAM-T | 5′-6-carboxyfluorescein-thymidine |
| ddG | 2′,3′-dideoxyguanosine |
| ddA | 2′,3′-dideoxyadenosine |
| ddC | 2′,3′-dideoxycytidine |
| ddT | 2′,3′-dideoxythymidine |
| (2′dF)A | 2′-deoxyfluoroadenosine |
| (2′dF)C | 2′-deoxyfluorocytidine |
| (2′dF)G | 2′-deoxyfluoroguanosine |
| (2′dF)U | 2′-deoxyfluorouridine |
| (2′dF)A-3′P | 2′-deoxyfluoroadenosine with a 3′-PO4 blocking group |
| (2′dF)C-3′P | 2′-deoxyfluorocytidine with a 3′-PO4 blocking group |
| (2′dF)G-3′P | 2′-deoxyfluoroguanosine with a 3′-PO4 blocking group |
| (2′dF)U-3′P | 2′-O-methyluridine with a 3′-PO4 blocking group |
| mA | 2′-O-methyladenosine |
| mC | 2′-O-methylcytidine |
| mG | 2′-O-methylguanosine |
| mU | 2′-O-methyluridine |
| mA-3′P | 2′-O-methyladenosine with a 3′-PO4 blocking group |
| mC-3′P | 2′-O-methylcytidine with a 3′-PO4 blocking group |
| mG-3′P | 2′-O-methylguanosine with a 3′-PO4 blocking group |
| mU-3′P | 2′-O-methyluridine with a 3′-PO4 blocking group |
| rA | adenosine ribonucleotide |
| rC | cytidine ribonucleotide |
| rG | guanosine ribonucleotide |
| rU | uridine ribonucleotide |
| (MOE)A | 2′-O-(2-methoxyethyl)adenosine |
| (MOE)C | 2′-O-(2-methoxyethyl)cytidine |
| (MOE)G | 2′-O-(2-methoxyethyl)guanosine |
| (MOE)U | 2′-O-(2-methoxyethyl)uridine |
| 5′-BiosG-T | thymidine with a 5′-biotin modifition |
| (iFluorT) | thymidine with a 5′-fluorescein modification |
| rA-3′P | adenosine ribonucleotide with a 3′-PO4 blocking group |
| rG-3′P | cytidine ribonucleotide with a 3′-PO4 blocking group |
| rC-3′P | guanosine ribonucleotide with a 3′-PO4 blocking group |
| rU-3′P | uridine ribonucleotide with a 3′-PO4 blocking group |
| dA-3′P | 2′-deoxyadenosine with a 3′-PO4 blocking group |
| dC-3′P | 2′-deoxycytidine with a 3′-PO4 blocking group |
| dG-3′P | 2′-deoxyguanosine with a 3′-PO4 blocking group |
| dT-3′P | 2′-deoxythymidine with a 3′-PO4 blocking group |
| A-3′P | 2′-deoxyadenosine with a 3′-PO4 blocking group |
| C-3′P | 2′-deoxycytidine with a 3′-PO4 blocking group |
| G-3′P | 2′-deoxyguanosine with a 3′-PO4 blocking group |
| T-3′P | 2′-deoxythymidine with a 3′-PO4 blocking group |

APPENDIX TABLE A2
Oligonucleotides included in sequence listing

| Oligonucleotide alias | SEQ ID NO |
|---|---|
| 5′-6-FAM-T17mAmUmC | 5129 |
| 5′-6-FAM-T17mAmUmCrA-3′P | 5130 |
| 5′-6-FAM-T17mAmUmCrG-3′P | 5131 |
| 5′-6-FAM-T17mAmUmCrC-3′P | 5132 |
| 5′-6-FAM-T17mAmUmCrU-3′P | 5133 |
| 5′-6-FAM-T17mAmUmC(2′dF)A-3′P | 5134 |
| 5′-6-FAM-T17mAmUmC*rG-3′P | 5135 |

Oligonucleotide modifications are described in Appendix Table A1.

1. A method of producing a nucleotide triphosphate with a phosphate group at the 3′ position of the sugar (NQP), the method comprising at least:
contacting a nucleoside diphosphate (NDP) with a nucleoside diphosphate kinase, a 3′-O-kinase, and a phosphate donor under reaction conditions such that a NTP with a phosphate group on the 3′ position of the sugar moiety (NQP) is produced.

2. The method of claim 1, wherein the NDP has at the 2′ position of the sugar moiety a H or OH.

3. The method of claim 2, wherein the NDP is ADP, GDP, UDP, CDP, or TDP, and wherein the NDP has at the 2′-position of the sugar moiety an OH, thereby resulting in corresponding product rATP, rGTP, rUTP, rCTP, or rTTP, respectively.

4. The method of claim 2, wherein the NDP is ADP, GDP, UDP, CDP, or TDP, and wherein the NDP has at the 2′-position of the sugar moiety a H, thereby by resulting in corresponding product dAMP, dGMP, dUMP, dCMP, or dTMP, respectively.

5. The method of claim 1, wherein the NDP is a modified NDP comprising a modified 2′-position, wherein the 2′-position of the sugar moiety is —O—CH₃, —O—CH₂CH₃, F, or Br.

6. The method of claim 1, wherein the NDP comprises a modified nucleobase, wherein the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

7. The method of claim 1, wherein the NDP comprises a 5′-O-(1-thio(diphosphate) (NDPαS).

8. The method of claim 1, further comprising contacting a nucleoside monophosphate (NMP) with a nucleoside monophosphate kinase in presence of a phosphate donor NTP under suitable reaction conditions such that a product nucleoside diphosphate (NDP) is produced for reaction with the nucleoside diphosphate kinase and/or 3′-O-kinase.

9. The method of claim 8, wherein the NMP has at the 2′ position of the sugar moiety a H or OH.

10. The method of claim 9, wherein the NMP is AMP, GMP, UMP, CMP, or TMP, and wherein the NMP has at the 2′-position of the sugar moiety an OH, thereby by resulting in corresponding product rADP, rGDP, rUDP, rCDP, or rTDP, respectively.

11. The method of claim 9, wherein the NMP is AMP, GMP, UMP, CMP, or TMP, and wherein the NMP has at the 2′-position of the sugar moiety a H, thereby by resulting in corresponding product dADP, dGDP, dUDP, dCDP, or dTDP, respectively.

12. The method of claim 8, wherein the NMP is a modified NMP comprising a modified 2′-position of the sugar moiety, wherein the 2′-position of the sugar moiety is —O—CH₃, —O—CH₂CH₃, F, or Br.

13. The method of claim 8, wherein the NMP comprises a modified nucleobase, wherein the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

14. The method of claim 8, wherein the NMP comprises a nucleoside 5′-O-thiophosphate (NMPαS).

15. The method of claim 8, wherein the phosphate donor NTP has the same nucleotide structure as the substrate NMP.

16. The method of claim 8, wherein the nucleoside monophosphate kinase is an adenylate kinase.

17. The method of claim 16, wherein the adenylate kinase is an adenylate kinase of an even-numbered SEQ ID NO. of SEQ ID NOs: 1206-2504, with or without amino acid residues 1-12.

18. The method of claim 8, further comprising contacting a nucleoside with a nucleoside kinase in presence of a phosphate donor NTP under reaction conditions such that a product nucleoside monophosphate (NMP) is produced.

19. The method of claim 18, wherein the nucleoside has at the 2′ position of the sugar moiety a H or OH.

20. The method of claim 19, wherein the nucleoside is adenosine (A), guanosine (G), uridine (U), cytidine (C), or thymidine (T), and wherein the nucleoside has at the 2′-position of the sugar moiety an OH, thereby by resulting in corresponding product rAMP, rGMP, rUMP, rCMP, or rTMP, respectively.

21. The method of claim 19, wherein the nucleoside is adenosine (A), guanosine (G), uridine (U), cytidine (C), or thymidine (T), and wherein the nucleoside has at the 2′-position of the sugar moiety a H, thereby by resulting in corresponding product dAMP, dGMP, dUMP, dCMP, or dTMP, respectively.

22. The method of claim 18, wherein the nucleoside is a modified nucleoside, wherein the 2′-position of the sugar moiety is —OCH₃, —OCH₂CH₃, F, or Br.

23. The method of claim 18, wherein the nucleoside comprises a modified nucleobase, wherein the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

24. The method of claim 18, wherein the phosphate donor NTP is a nucleoside-5′-gamma thio-triphosphate (NTPγS) for producing NMP-5′-O-thiophosphate (NMPαS).

25. The method of claim 18, wherein the nucleoside kinase is an adenosine kinase.

26. The method of claim 25, wherein the adenosine kinase is an adenosine kinase of an even-numbered SEQ ID NO. of SEQ ID NOs: 66-1204, with or without amino acid residues 1-12.

27. The method of claim 1, wherein the nucleoside diphosphate kinase is an acetate kinase.

28. The method of claim 27, wherein the acetate kinase is an acetate kinase of an even-numbered SEQ ID NO. of SEQ ID NOs: 2506-3058, with or without amino acid residues 1-12.

29. The method of claim 1, wherein the 3′-O-kinase is a 3′-O-kinase of an even-numbered SEQ ID NO. of SEQ ID NOs: 3060-3370 and 3376-5126, with or without amino acid residues 202-211.

30. A method of producing a NTP with a phosphate group at the 3′ position of the sugar (NQP), the method comprising contacting a nucleoside with a nucleoside kinase, a nucleoside monophosphate kinase, nucleoside diphosphate kinase, and a 3′-O-kinase in presence of a phosphate donor under reaction conditions such that a product NTP with a phosphate group at the 3′ position of the sugar (NQP) is produced.

31. A method of producing a NTP with a phosphate group at the 3′ position of the sugar (NQP), the method comprising (i) contacting a nucleoside with a nucleoside kinase, a nucleotide monophosphate kinase, and a nucleoside diphosphate kinase in presence of a phosphate donor under reaction conditions such that a NTP is produced; and iii) contacting the NTP produced in (i) with a 3′-O-kinase enzyme under suitable reaction conditions, such that a NTP with a phosphate group at the 3′ position of the sugar (NQP) is produced.

32. A method of producing a NTP with a phosphate group at the 3′ position of the sugar (NQP), the method comprising (i) contacting a nucleoside with a nucleoside kinase, (ii) contacting the product of (i) with a nucleotide monophosphate kinase, (iii) contacting the product of (ii) with a nucleoside diphosphate kinase, and (iv) contacting the product of (iii) with a 3′-O-kinase enzyme, wherein (i), (ii), (iii) and (iv) are carried out in presence of a phosphate donor under reaction conditions such that a product NTP with a phosphate group at the 3′ position of the sugar (NQP) is produced.

33. The method of claim 30, wherein the nucleoside has at the 2′-position of the sugar moiety a H or OH.

34. The method of claim 33, wherein the nucleoside is adenosine (A), guanosine (G), uridine (U), cytidine (C), or thymidine (T), and wherein the nucleoside has at the 2′-position of the sugar moiety an OH, thereby by resulting in corresponding product rAQP, rGQP, rUQP, rCQP, or rTQP, respectively.

35. The method of claim 33, wherein the nucleoside is adenosine (A), guanosine (G), uridine (U), cytidine (C), or thymidine (T), and wherein the nucleoside has at the 2′-position of the sugar moiety a H, thereby by resulting in corresponding product dAQP, dGQP, dUQP, dCQP, or dTQP, respectively.

36. The method of claim 30, wherein the nucleoside comprises a modified 2′-position of the sugar moiety, wherein the 2′-position of the sugar moiety is —OCH₃, —OCH₂CH₃, F, or Br.

37. The method of claim 30, wherein the nucleoside comprises a modified nucleobase, wherein the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

38. The method of claim 30, wherein the phosphate donor comprises phosphate donor NTP.

39. The method of claim 38, wherein the phosphate donor NTP is a nucleoside-5′-gamma thiotriphosphate (NTPγS).

40. The method of claim 30, wherein the nucleoside kinase is an adenosine kinase.

41. The method of claim 40, wherein the adenosine kinase is an adenosine kinase of an even-numbered SEQ ID NO. of SEQ ID NOs: 66-1204, with or without amino acid residues 1-12.

42. The method of claim 30, wherein the nucleoside monophosphate kinase is an adenylate kinase.

43. The method of claim 42, wherein the adenylate kinase is an adenylate kinase of an even-numbered SEQ ID NO. of SEQ ID NOs: 1206-2504, with or without amino acid residues 1-12.

44. The method of claim 30, wherein the nucleoside diphosphate kinase is an acetate kinase, and the phosphate donor includes acetyl phosphate.

45. The method of claim 44, wherein the acetate kinase is an acetate kinase of an even-numbered SEQ ID NO. of SEQ ID NOs: 2506-3058, with or without amino acid residues 1-12.

46. The method of claim 30, wherein the 3′-O′-kinase comprises a 3′-O′-kinase of an even-numbered SEQ ID NO. of SEQ ID NOs: 3060-3370 and 3376-5126, with or without amino acid residues 202-211.

47-70. (canceled)

71. A method of producing a nucleoside triphosphate (NTP), comprising contacting a nucleoside with a nucleoside kinase, a nucleoside monophosphate kinase, and a nucleoside diphosphate kinase in presence of a phosphate donor under reaction conditions such that NTP is produced.

72-86. (canceled)

87. The method of claim 1, further comprising a NTP regenerating system.

88. The method of claim 87, wherein the NTP regenerating system comprises creatine kinase, pyruvate kinase, polyphosphate kinase, and/or R-acetate-kinase.

89. The method of claim 88, wherein the NTP regenerating system is pyruvate kinase and substrate phosphoenolpyruvate.

90. The method of claim 88, wherein the NTP regenerating system is creatine kinase and substrate creatine phosphate.

91. The method of claim 88, wherein the NTP regenerating system is polyphosphate kinase and substrate polyphosphate.

92. The method of claim 88, wherein the NTP regenerating system is R-acetate kinase and the substrate acetyl phosphate.

93. The method of claim 92, wherein the R-acetate kinase is same or different than the acetate kinase of any one of claims 27, 28, 44, 45, 69, 70, 85, and 86.

94. The method of claim 92, further comprising pyruvate oxidase and substrate pyruvate and inorganic phosphate.

95. The method of claim 94, further comprising a catalase.
